SEQUENCE LISTING (10 sequences)

SEQ ID NO: 1 - DNA, 20 bp, Artificial Sequence
  TRPC1 Forward Primer
aagcttttcttgctggcgtg

SEQ ID NO: 2 - DNA, 20 bp, Artificial Sequence
  TRPC1 Reverse Primer
atctgcagactgacaaccgt

SEQ ID NO: 3 - DNA, 20 bp, Artificial Sequence
  SNAIL Forward Primer
cgagtggttcttctgcgcta

SEQ ID NO: 4 - DNA, 22 bp, Artificial Sequence
  SNAIL Reverse Primer
ctgctggaaggtaaactctgga

SEQ ID NO: 5 - DNA, 22 bp, Artificial Sequence
  SLUG Forward Primer
tagaactcacacgggggagaag

SEQ ID NO: 6 - DNA, 20 bp, Artificial Sequence
  SLUG Reverse Primer
attgcgtcactcagtgtgct

SEQ ID NO: 7 - DNA, 20 bp, Artificial Sequence
  Vimentin Forward Primer
aaggcgaggagagcaggatt

SEQ ID NO: 8 - DNA, 20 bp, Artificial Sequence
  Vimentin Reverse Primer
aggtcatcgtgatgctgaga

SEQ ID NO: 9 - DNA, 25 bp, Artificial Sequence
  Beta Actin Forward Primer
agaagatgacccagatcatgtttga

SEQ ID NO: 10 - DNA, 20 bp, Artificial Sequence
  Beta Actin Reverse Primer
agcacagcctggatagcaac

(19) United States
(12) Patent Application Publication
(10) Pub. No.: US 2023/0372244 A1
(43) Pub. Date: Nov. 23, 2023

(54) Methods of Preparing Cell-Derived Vesicles

(71) Applicants: NATIONAL UNIVERSITY OF SINGAPORE, Singapore (SG); ETH Zürich, Zürich (CH)

(72) Inventors: Alfredo Franco-Obregon, Singapore (SG); Yee Kit Tai, Singapore (SG); Felix Kurth, Zurich (CH); Petra Stephanie Dittrich, Basel (CH)

(21) Appl. No.: 18/030,535

(22) PCT Filed: Oct. 12, 2021
(86) PCT No.: PCT/SG2021/050613

§ 371 Date: Apr. 6, 2023

(30) Foreign Application Priority Data
Oct. 12, 2020 (SG) 10202010091U

(51) Int. Cl. A61K 9/127; C12N 5/09; C12N 13/00; A61K 38/17; A61P 35/00

(52) U.S. Cl. CPC A61K 9/127; C12N 5/0693; C12N 13/00; A61K 9/1277; A61K 38/177; A61P 35/00; C12N 2509/00

(57) ABSTRACT

The invention relates generally to cell-derived vesicles (CDVs), such as CD Vs from myoblast, wherein the parent cells have been subjected to pulsed electromagnetic field and wherein the vesicles are isolated from the parent cells using actin cytoskeletal disruptors, such as cytochalasin B. The CDVs are enriched for Transient Receptor Potential Channel 1 (TRPC1). The invention further relates to the uses of the CDVs as therapeutic agents in conditions associated with disruptive metabolism and inflammation; for instance, cancer.

### FIELD OF INVENTION

The invention relates generally to the field of cell-derived vesicles. In particular, the invention relates to cell-derived vesicles as Transient Receptor Potential Channel 1 (TRPC1) channel delivery systems.

### BACKGROUND

Mitochondria are membrane-bound organelles found in the cytoplasm of nucleated eukaryotic cells. They are found in almost every cell of the human body except red blood cells. They are the cell's primary site of energy metabolism and generate adenosine triphosphate (ATP) for different cell functions. Typically, more than 90% of a cell's requirement for ATP is supplied by the cell's own mitochondria.

Mitochondrial function is key to human health, longevity and regenerative capacity. Due to mitochondria's primary function in cell metabolism, damage and dysfunction in mitochondria can cause a wide range of human diseases. There are currently, however, no known and approved treatments that involves mitochondria.

Transient Receptor Potential Channel 1 (TRPC1) channels are ubiquitously linked to tissue developmental programs and regenerative cycles. TRPC1 is the most widely expressed of all TRP channels. TRPC1 is capable of stimulating mitochondrial respiration, which also gives it a regenerative function. The dysregulation of TRPC1 also contributes to different types of cancer. There are, however, no known treatments that promote the activity of TRPC1 channels in subjects.

Accordingly, it is generally desirable to overcome or ameliorate one or more of the above mentioned difficulties.

### SUMMARY

Disclosed herein is a method of preparing cell-derived vesicles from a eukaryotic cell, the method comprising:
a) exposing a cell to a pulsed electromagnetic field (PEMF) to induce Transient Receptor Potential Channel 1 (TRPC1) protein expression or activity in the cell;
b) contacting the cell with an actin cytoskeletal disruptor under mixing conditions to promote release of cell-derived vesicles from the cell surface; and
c) isolating the cell-derived vesicles.

Disclosed herein is a population of cell-derived vesicles obtained according to a method as defined herein.

Disclosed herein is the use of a population of cell-derived vesicles as defined herein for delivering TRPC1 to a subject.

Disclosed herein is the use of a population of cell-derived vesicles as defined herein as a biosensor.

Disclosed herein is a pharmaceutical composition comprising cell-derived vesicles obtained according to a method as defined herein.

Disclosed herein is a method of promoting cellular or tissue regeneration in a subject, wherein the method comprises administering a population of cell-derived vesicles or a pharmaceutical composition as defined herein to the subject.

Disclosed herein is a method of treating a disease or condition in a subject, wherein the method comprises administering a population of cell-derived vesicles or a pharmaceutical composition as defined herein to the subject.

Disclosed herein is a method of sensitizing a subject to an anti-cancer therapy, the method comprising administering a population of cell-derived vesicles or a pharmaceutical composition as defined herein to the subject in combination with the anti-cancer therapy.

### BRIEF DESCRIPTION OF THE FIGURES

FIG. 1. Schematic depicting the tests applied to CDVs to ascertain 1) innate channel functionality and 2) recuperative potential for cellular respiratory and proliferative capacities. 1) Cell-free assays were comprised of examining specific TRP channel activation by menthol (TRPA1) or magnetic fields (TRPC1). 2) Cell-based assays entailed administering wild type CDVs to TRPC1-knockdown myoblasts followed by testing for recuperation of cellular proliferative and mitochondrial respiratory capacities.

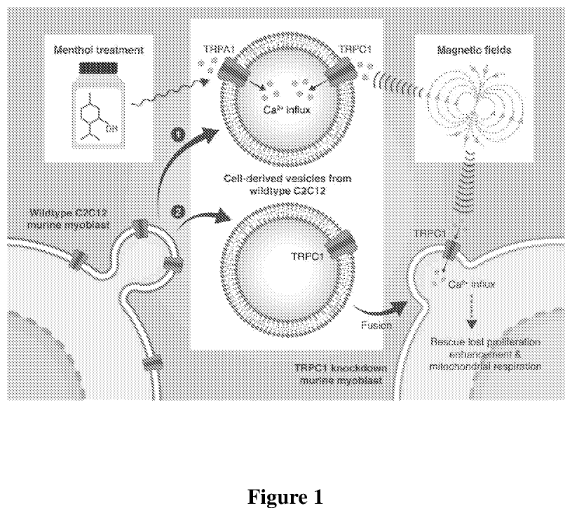

FIG. 2. CDVs derivation from myoblast and myotube cultures. A, left) CDV yield increased with increasing myoblast density and in myotube cultures differentiated from confluent myoblast cultures. A, right) Size distributions of CDVs derived from myoblast and myotube cultures. The thick dotted line indicates the median value and the thin dotted lines the quartile values. Mean diameters were not statistically different (n.s.: not significant) between CDV samples from myoblast and myotube cultures. CDVs were generated from 8 and 5 independent myoblast and myotube cultures, respectively, A, left and right). B) CDV size frequency distribution before (left) and after extrusion (right) with a 200 nm diameter pore size. C) Freeze fracture SEM of a non-extruded CDV sample. Asterisks (*) identify liposomes that partially look out of the surface, hashtags (#) depict imprints within the surface from CDVs that were entirely detached during the fracture process thereby leaving a crater in the surface. Arrows reveal smaller vesicular bodies that were enclosed within the lumens of larger liposomes during the CDV formation. Scale bar: 1 D) TEM micrograph of extruded CDVs shown in B (right). The blue coloured area depicts the free carbon grid. The black triangles mark the lipid bilayer with a diameter of 4-5 nm; individual monolayers are visible as thin dark lines. The 2 CDVs adjacent to the free carbon grid are deformed due to the limited height of the sample holder. Scale bar: 100 nm. Panels B, C, and D depict data generated from myotube-derived CDVs.

FIG. 3. A) Proteome analysis of CDVs and its comparison to published data. Comparison of GO-assigned fractions of identified proteins from C2C12 myotube-derived CDVs and published intact C2C12 myotubes. CDVs exhibit relative enrichments of protein identifications with GO terms associated with membrane and the extracellular region. B) The percentage of total identified proteins for GOs of CDV samples provides an overview of the cell component protein associations. C) Chromatographic traces (PRM measurements) of fragment ions matching proteotypic peptides from the respective calcium channels TRPC1, ORM1, and TRPA1. Grey asterisks mark the relevant signals. D) Lipid abundances for CDVs and C2C12 myotubes. E) Comparison of lipid abundances between CDVs and C2C12 myotubes. The most significant changes in lipid content (CDVs relative myotubes) originates from the sphingomyelins (+5.99%) and phosphatidylserines (+12.5%) as well as from phosphatidylcholines (−18.51%). A list of the detailed concentrations of lipid components in CDVs and whole cell samples is available in the appendix. Lipid abbreviations are: TG: triglyceride, SM: sphingomyelin, PS: phosphatidylserine, PI: phosphatidylinositol, PE: phosphatidylethanolamine, PC: phosphatidylcholine, LPC: lysophosphatidylcholine, CER: Ceramide.

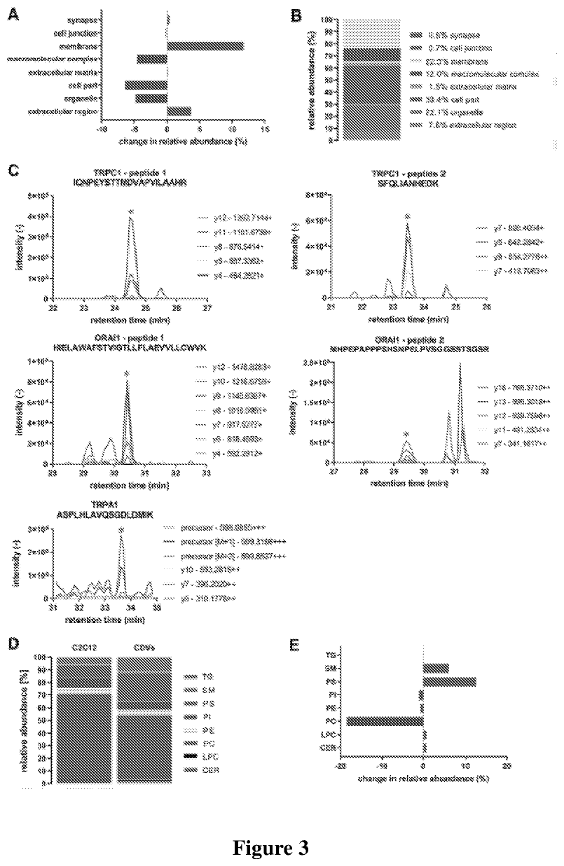

FIG. 4. A) Schematics of the CDV immobilization 1), loading 2), and TRP channel functional assessment 3). B) Micrographs of the print of the biochemical cholesterol linkers (left) and CDVs immobilized on the same print and loaded with calcein-AM. Red fluorescence is Texas Red as part of the biochemical linker, the dotted line indicates the edge of the print. Scale bar: 20 μm. C) Menthol-induced increases in intra-CDV calcium concentration (red trace) compared to the signal from CDVs not administered menthol (black trace). Data were simultaneously recorded from 3 parallel channels of the same microfluidic chip containing Fluo-4 loaded myoblast-derived CDVs from the same culture, 2 channels with menthol administration and one without (negative control). The menthol signal represents the average±standard deviation (shaded area). D, E) TRPC1-mediated calcium uptake in response to magnetic stimulation. Representative calcium responses (Rate of Rise (ROR) normalized to starting value) from intact C2C12 myoblasts D) or CDVs E) derived from wild type C2C12 myoblasts (left) or C2C12 CRISPR/Cas9 knockdown myoblasts, 60 (middle), and 61 (right), in response to 10 min magnetic field exposure (1.5 mT) while in suspension immediately before reading. Blue and red lines represent magnetically-exposed and control samples, respectively, as indicated. F) Mean calcium responses from CDVs derived from C2C12 wild types (left) or knockdown clones 60 (middle) and 61 (right) as indicated. All values are shown normalized to C2C12 CDVs at time 0 min and 0 mT. CDVs were loaded with Calcium Green-1 AM (0.5 ng μL−1) for 30 min, while in culture followed by preparation of CDVs for subsequent magnetic stimulation and immediate analysis. Red shaded area reflects inhibition relative to C2C12 CDVs at time 0 min and 0 mT. Data represent the means of 6-11 independent experiments (biological replicates), each representing the means of 8-10 technical replicates. ** and * represent P<0.01 and <0.05 relative to respective 0 mT at same time point (blue) or to time=0 min in C2C12 CDVs (red) as indicated. Statistical significance was not achieved in either TRPC1-knockdown clone, 60 or 61, relative to their respective 0 mT or 0 min conditions. All CDVs were derived from myoblasts.

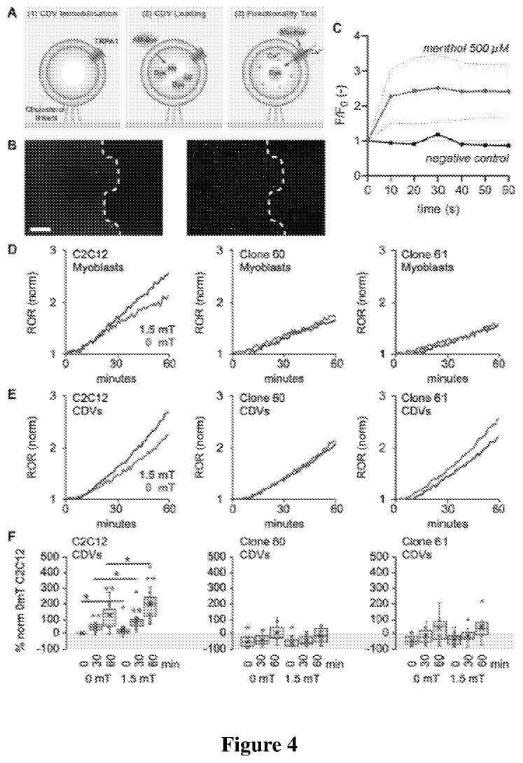

FIG. 5. CDV-mediated rescue of magneto-reception in TRPC1 knockdown myoblasts. A) Representative magnetically-stimulated proliferative responses of C2C12 wild type (top), c60 TRPC1 knockdown (KD; middle), and c61 TRPC1 knockdown (KD; bottom) myoblast cultures with (+CDV) and without (−CDV) the addition of CDVs derived from C2C12 wild type cultures as indicated. CDVs were added to myoblast cultures 8 h post-cell seeding and 16 h before magnetic exposure at an amplitude of 1.5 mT for 10 min as previously described. Culture DNA content (Cyquant) was measured 24 h after magnetic exposure. Blue and red bars indicate magnetically-exposed and non-magnetically exposed control samples, respectively (**P<0.01 with regards to respective 0 mT). n=12 wells per condition. B) Magnetically-induced proliferation responses (1.5 mT/0 mT) for wild type C2C12 myoblasts (left) and CRISPR/Cas9 knockdown C2C12 myoblast clones, c60 (middle) and c61 (right) with (blue) and without (orange) the addition of wild type CDVs derived from either myoblasts (middle), myotubes (bottom) or both combined (top) as indicated. Per condition, the data depicted represents the means of 12 independent experiments (biological replicates), each representing the means of n=10-12 technical replicates. ** and * represent P<0.01 and <0.05, respectively, pairwise for 0 versus 1.5 mT A) or −CDV versus +CDV B).

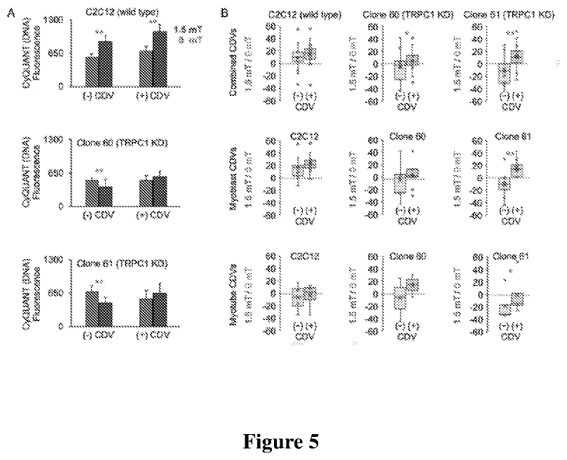

FIG. 6. Relative changes [(1.5/0 mT) plus wt CDVs/(1.5/0 mT) minus wt CDVs] in pulsing magnetic fields (PEMF)-induced oxygen consumption rate upon the addition of wild type myoblast CDVs. Changes in PEMF-induced Basal Respiration A), Spare Capacity B), and ATP Production C) for CRISPR/Cas9 knockdown C2C12 clones, c60 (light blue), and c61 (dark blue) following the administration of wild type myoblasts CDVs (+CDVs) relative to untreated cells (—CDVs). Red shaded boxes indicate areas of no or negative change in PEMF response following CDV addition. Data represent the means of 7 independent experiments (biological replicates), each representing the means of technical triplicates. (*P<0.05 relative to untreated cells of the same origin).

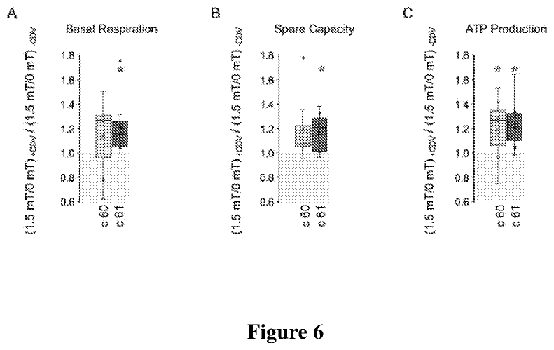

FIG. 7. Downwardly directed magnetic fields specifically-enrich CDVs with TRPC1 channels and confer biological activity. Results obtained from CDVs generated 6 h (A, C, E) and 24 h (B, D, F) following magnetic exposure. Growth of recipient myoblasts receiving CDVs-derived from myoblasts that have been magnetically exposed to 1.5 mT downward field (C) and (D), or upward field (E) and (F). The estimation plots in (C) and (E) correspond to CDVs collected 6 h post magnetic stimulation while (D) and (F) correspond to CDVs after 24 h magnetic stimulation. The bar charts in (A) and (B) show the relative protein expression of TRPC1 channel in CDVs analyzed 24 h and 6 h post magnetic stimulation, respectively. Statistical analysis was done using Ordinary one-way ANOVA with Sidak's multiple comparison test, with *p<0.05 and the error bars represent the standard error of the mean.

FIG. 8. Loading of anti-cancer proteins in CDVs-derived from myoblasts 6 h post magnetic stimulation in the downward field direction. The bar charts show the pooled data of full length HTRA1 (A), cleaved HTRA1 (B) and Klotho (C) protein expression in CDVs-derived from myoblasts post 6 h magnetic stimulation in the downward field direction, compared to unexposed 0 mT. Insets show protein expression in CDVs post 24 h magnetic stimulation. (D) Presence of secreted HTRA1 in conditioned media from myotubes that have been exposed to magnetic fields. (E) Pool data showing the relative live cell count of MCF-7 breast cancer cells in response to 5 nM recombinant HTRA1. (F) Bar chart shows the pooled data for cellular proliferation of MCF-7 breast cancer cells after administration of conditioned media containing secreted HTRA1 protein from constitutively overexpressing cell line. Clones 2 and 12 were generated from single cell sorting and antibiotic selection. (G) Bar chart represents the pooled data for MCF-7 proliferation 24 h after incubation in non-exposed or PEMF-exposed myoblast-derived CDVs. Delivery of CDVs stimulated the killing of breast cancer cells that would be further enhanced with subsequent PEMF exposure alone and in combination with doxorubicin chemotherapy. Statistical analysis using Students t-test were used to compare two sample means. One-way ANOVA was used to analyze (F), with Sidak's multiple comparison test. * p<0.05 and ** p<0.01 with the error bars representing the standard error of the mean.

FIG. 9 showing the generation of downward magnetic fields specifically-enrich CDVs with TRPC1 channels and confer biological activity. A coil configuration was used to create circular/oval continuous field lines traveling through the coil centre where they concentrate uniformly to create the specified fields. The field lines substantially coincide with the direction of the Earth's gravitational field.

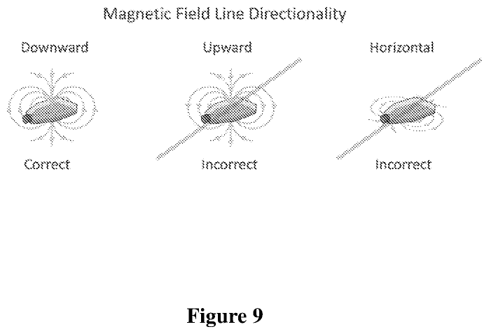

FIG. 10. PEMFs synergize with DOX to inhibit tumor growth in vivo. (A) Schematic of PEMF and DOX exposures regimes used on mice hosting patient-derived tumor xenografts. Implanted tumors were allowed to grow for 3 weeks before the initiation of DOX and/or PEMF treatments. Tumor volumes were measured each week while apoptotic cell determination was performed at the end of the study. Each data point represents the mean from 5 experimental runs derived from the tumors obtained from 5 patients, each of which was equally divided amongst the 5 treatment groups. (B) Changes in tumor volume (mm³) for 5 weeks. (C) Representative scatter dot-plots showing cell population of dissociated tumors sorted based on annexin and propidium iodide staining. (D) Bar chart represents pooled data of apoptotic cell percentages analyzed using flow cytometry. N=5 mice, with *, p<0.05, **, p<0.01, and #p<0.0001. The error bars are expressed as the standard error of the mean.

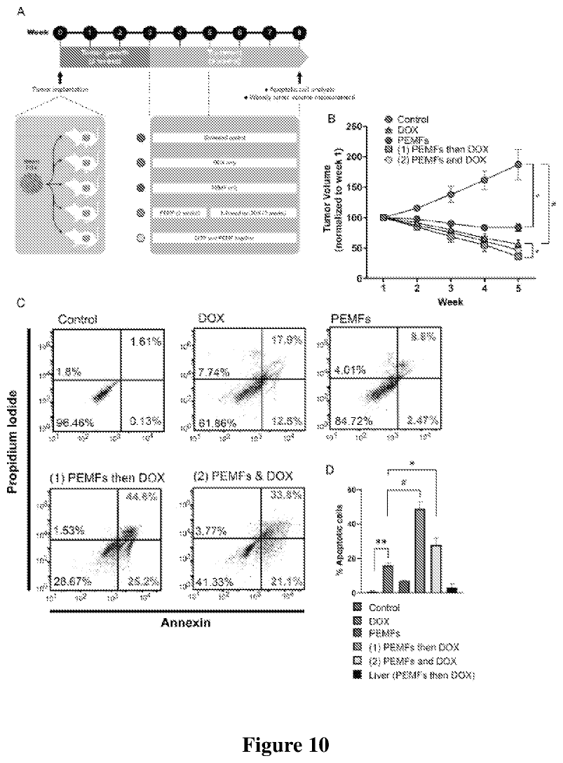

FIG. 11. PEMF inhibits breast tumor growth in vivo correlated with depressed TRPC1 expression levels (A) Schematic of the PEMF exposure paradigm used on the CAM model for MCF-7 breast tumor xenografts. MCF-7 tumors were inoculated onto CAM on day 7. The tumors were either exposed to 3 mT for 1 h for 3 successive days or exposed to 3 mT after 1 h of DOX treatment, both starting from day 10 onwards. (B) Bar chart shows the weight of chick embryo analyzed on final day 14. (C) Representative images showing the size of MCF-7 tumors at day 14 and the corresponding bar chart in (D) represents the fold change in tumor weight over 0 mT. Bar charts in (E) and (F) show the pooled data of TRPC1 and Cyclin D1 protein expression levels normalized to GAPDH, respectively, expressed as fold change over 0 mT. (G) Representative images showing MCF-7 tumor size after treatment with 0 mT with saline, 0 mT with DOX and 3 mT with DOX. The pooled data of tumor weight (H) is expressed as fold change over 0 mT. The bar charts in (I) and (J) represent the pooled data of TRPC1 and Cyclin D1 protein expression levels normalized to GAPDH, expressed as fold change over 0 mT with saline, respectively. Experiments were repeated at least thrice with *, p<0.05 and **, p<0.01 of at least 6 independent eggs. The error bars represent the standard error of the mean. (K) Representative micrographs of 5 chick livers per group showing TUNEL staining following PEMF exposure and in controls. Scale bar=100 μm. The liver sections as positive controls were treated with DNAse prior to the staining with TUNEL. The nuclei were stained with DAPI (blue).

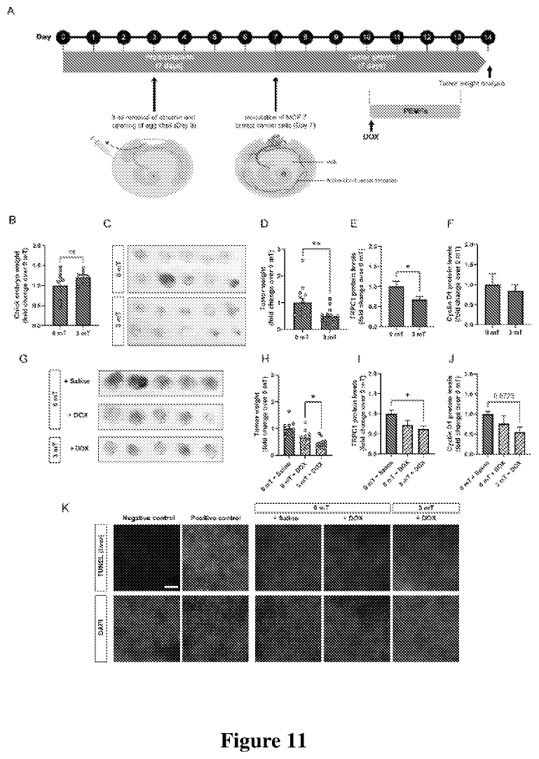

FIG. 12. PEMF exposure inhibits cancer cell growth in vitro and ex vivo (human). (A) Schematic of PEMF exposure schedule for live cell quantification. (B), (C), and (D) Live cell counts using Trypan Blue exclusion assay for MCF-7, MDA-MB-231 and MCF10A cells. Cells were exposed to 0 mT, 3 mT, or 5 mT PEMF for 1 h each day for 3 consecutive days before cell counts were performed. (E) Schematic of MCF-7 cell colony formation regime. PEMF exposure was conducted at 3 mT for 1 h/day. (F) Representative images of MCF-7 cell colonies formed with and without PEMF exposure as indicated. Cells were seeded at a density of 100 per well. Quantification of surviving colonies presented as fold change over 0 mT. (G) Image of colony formation assay and the colony size frequency distribution, normalized to the total number of colonies. All experiments were from 3 to 6 independent biological replicates performed with *, p<0.05, **, p<0.01, ***, p<0.001. The error bars represent the standard error of the mean. (H) Representative images showing breast tumor biopsies and healthy breast tissues stained with TUNEL (green) 24 h post 3 mT PEMF exposure. Nuclei were stained with DAPI (blue). Scale bar=100 μm. The corresponding line plot shows the paired analysis between 0 mT and 3 mT of TUNEL staining per tissue area. Each line represents one independent patient sample, analyzed using one-tail paired t-test, comparing between 0 mT and 3 mT. Inset graph is the pooled average of the same TUNEL analysis after the normalization of 3 mT/0 mT of every individual sample, expressed as fold change over 0 mT. Analysis was performed using 2 way ANOVA with Sidak's multiple comparisons test, with *, p<0.05 and the error bars represent the standard error of the mean.

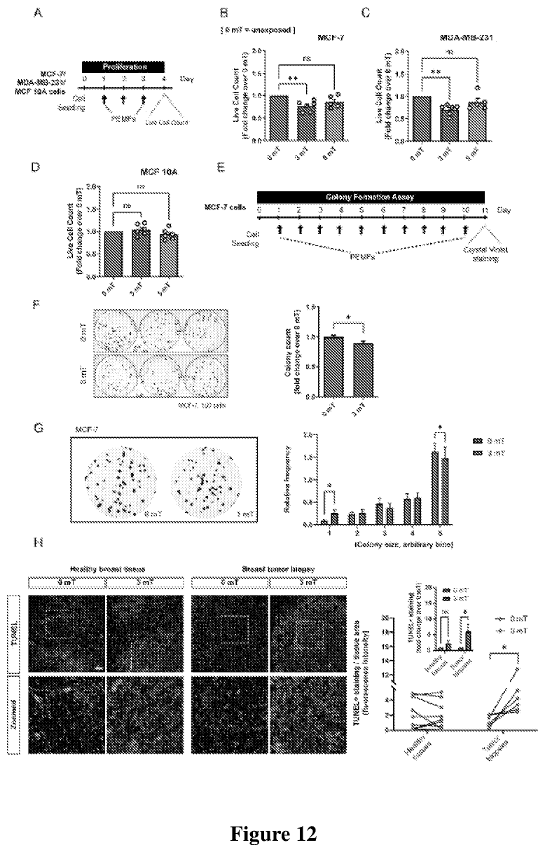

FIG. 13. PEMF exposure enhances MCF-7 cell vulnerability to doxorubicin. (A) Schematic of PEMF and DOX treatment regime for DNA quantification. Cells were exposed to 3 mT for 1 h daily for 3 successive days. DOX (100 nM) was administered on the final day 1 h before the last PEMF exposure. Cellular DNA content was measured 24 h after the last PEMF exposure. (B) Bar chart of pooled data for Cyquant DNA content in fold change 24 h post-DOX and PEMF treatments. (C) Colony formation paradigm for MCF-7 cells treated with DOX and PEMFs. (D) MCF-7 colony formation in the presence of 10 and 20 nM DOX, with and without PEMF exposure as indicated. Cells were seeded at a low density of 100 cells per well. (E) Colony survival in 10 nM DOX under low-density conditions. Colony number in the 20 nM DOX condition were too few to quantify accurately. (F) MCF-7 colony formation in the presence of 10 and 20 nM DOX, with and without PEMF exposure under high-density seeding of 1000 cells per well. The images on the right are the zoom-in images of cells under the same treatment paradigm. (G) The bar chart corresponds to the normalized colony size frequency distribution in the presence of 20 nM DOX under high-cell density condition, with 0 mT and 3 mT marked by hatched red and hatched blue bars, respectively. (H) Absolute DCH₂FDA-ROS fluorescence from MCF-7 cells. Cells were incubated in DCH₂FDA for 30 min before washing and replacement with media containing DOX or TBH. Cells were exposed to 3 mT PEMFs for 10 min followed by ROS measurement for 20 min. ROS values represent the average of 8 technical replicates per condition. All data presented were from 3 to 5 independent experiments, with *, p<0.05, **, p<0.01, ***, p<0.001, #p<0.0001. The error bars represent the standard error of the mean.

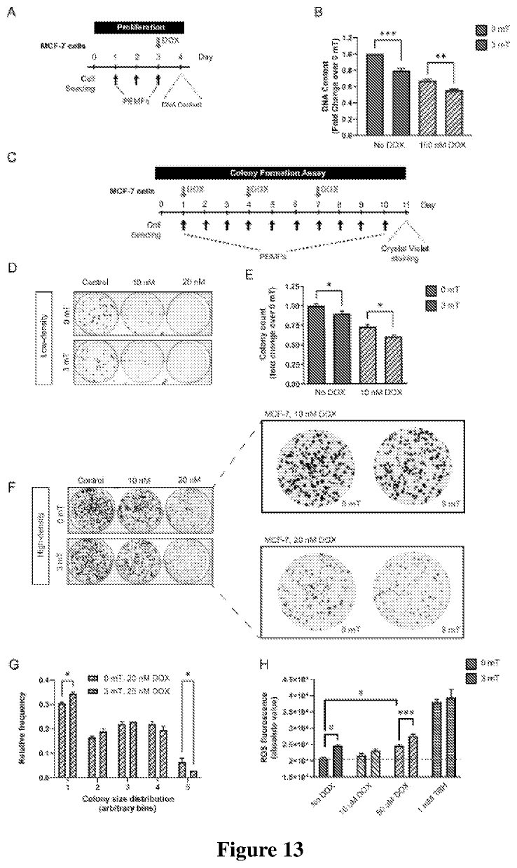

FIG. 14. DOX-chemoresistance is associated with TRPC1 channel downregulation. (A) PEMF (black arrows, 3 mT) and DOX (red arrow, 20 nM) regime used for TRPC1 protein analysis. (B) Representative western blot showing the changes in TRPC1 levels after PEMF and DOX treatments after 11 days. The corresponding bar chart shows TRPC1 protein levels normalized to unexposed 0 mT. Cells treated with DOX are represented by the hatched bars in combination with either 0 mT (red) or 3 mT (blue) PEMF exposure. (C) Western blot analysis of TRPC1 protein levels in MCF-7/ADR (96 nM DOX; gray) and 10-day 20 nM DOX-treated MCF-7 cells (hatched red) relative to naïve MCF-7 cells (red). Naïve MCF-7 and MCF-7/ADR cells were grown in culture for 3 days before protein analysis. (D) Comparison of cell growth (72 h post-seeding) and TRPC1 transcript levels between MCF-7/ADR (96 nM), MCF-7/ADR (0 nM) and naïve MCF-7 cells. MCF-7/ADR (96 nM) cells (also in FIG. 5C) were generated using incremental DOX exposures up to a concentration of 96 nM (gray). MCF7/ADR (96 nM) cells were serially passaged in the absence of DOX to give rise to MCF-7/ADR (0 nM) cells (yellow). (E) Western analysis of TRPC1 proteins levels in non-malignant (MCF10A) and malignant breast cancer (MCF-7 and MDA-MB-231) cells after 48 h of growth under standard conditions. The corresponding bar chart shows the pooled data in fold change of TRPC1 expression normalized to MCF10A. All results presented were from 3 to 5 independent experiments with *, p<0.05, **, p<0.01, ***, p<0.001, and #, p<0.0001. The error bars represent the standard error of the mean.

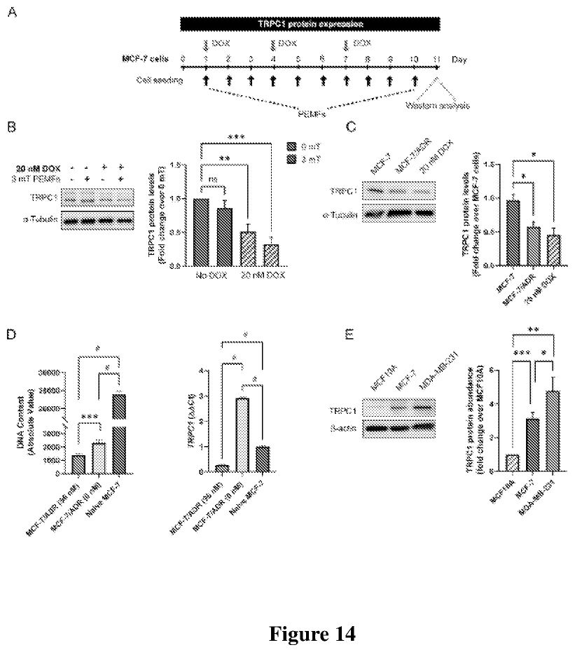

FIG. 15. Characterization of TRPC1 overexpressing MCF-7 cell line. (A) Western analysis showing the overexpression of GFP-TRPC1 in TRPC1 cells, stained using anti-GFP antibody. (B) Bar chart showing ΔΔCt fold change of TRPC1 transcript in MCF-7/TRPC1 cells (green) and vector-transfected cells (black). (C) Fluorescence images showing GFP and GFP-TRPC1 in vector and MCF-7/TRPC1 cells, respectively. Scale bar=10 μm. (D) Live cell counts for MCF-7/TRPC1 (green) and vector-transfected (black) cells over 3 days. (E) Western analysis showing cyclin D1 protein levels 24 h post-seeding. (F) Representative images of the migration assay over 4 days. Stable cells were seeded at a density of 30,000 per well of the culture insert one day before the removal of the insert to create a 0.5 mm gap. (G) Gap remaining 4 days into the migration assay. (H) Transcript levels of TRPC1, SLUG, SNAIL, VIMENTIN, and E-CADHERIN in vector and MCF-7/TRPC1 cells. (I) and (J) Representative confocal images of vector and MCF-7/TRPC1 cells stained for Vimentin and E-Cadherin alongside corresponding bar charts of mean intensity per cell. Absolute fluorescence intensity was normalized to the total number of nuclei per view. (K) Transcript levels for TRPC1, SLUG, SNAIL, VIMENTIN, and E-CADHERIN in scrambled- and TRPC1-silenced cells. (L) Proliferation over 3 days for TRPC1-silenced cells relative to scramble RNA-transfected cells. TRPC1 silencing was achieved using 2 independent dsiRNAs and the bar charts show the pooled data from the respective experiments. All results were from 3 to 5 independent experiments with *, p<0.05, **, p<0.01, ***, p<0.001, #, p<0.0001. The error bars represent the standard error of the mean.

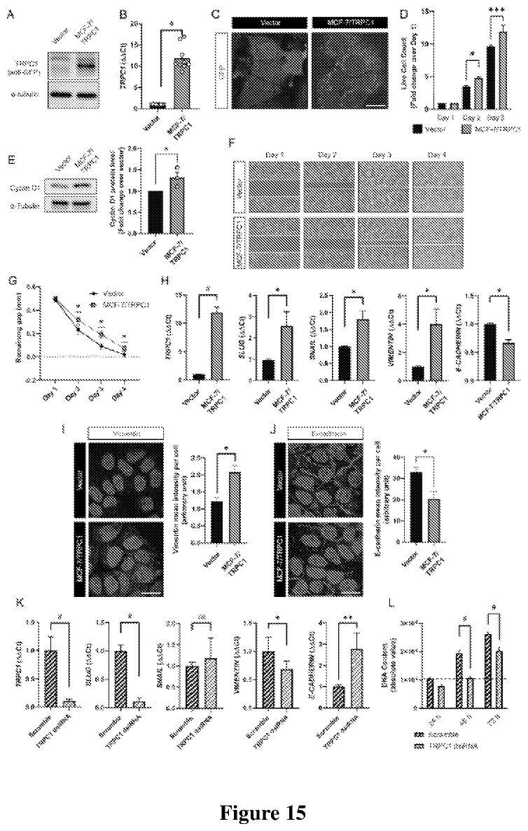

FIG. 16. PEMF exposure attenuates migration and invasion of MCF-7/TRPC1 cells. (A) Photographic comparison of the migration of vector-transfected and MCF-7/TRPC1 cells exposed to 0 or 3 mT PEMFs. Cells were plated at a density of 30,000 per well of the culture insert and allowed to settle for 24 h before the removal of the insert. Cells were exposed to PEMFs for 1 h on the second, third and fourth days. Pooled responses from (B) MCF-7/TRPC1 (0 and 3 mT) and (C) vector (0 and 3 mT) cells showing the remaining gap normalized to day 1. (D) Quantification of stained (invading) cells at the bottom of the basal membrane expressed as fold change relative to vector cells. The stained cells correspond to those successfully invading the basal membrane after 48 h. Untreated vector cells served as a reference of basal cell invasion (black). The second (solid gray) and fourth (hatched gray) bar show vector cells that had been treated with TGFβ during seeding to promote invasion. The hatched bars correspond to cells exposed to 3 mT PEMF at seeding and 24 h later. (E) Cell density on the upper insert of the chamber after 48 h post-seeding. The cells were stained and lysed using the same schedule as for the invasion assay. (F) Western analysis of E-cadherin protein expression in vector and MCF-7/TRPC1 cells with and without PEMF exposure for 3 consecutive days. E-cadherin protein fold change relative to 0 mT of vector cells. All results were from 3 to 5 independent experiments with *, p<0.05, **, p<0.01, #, p 1062<0.0001. The error bars are expressed as the standard error of the mean.

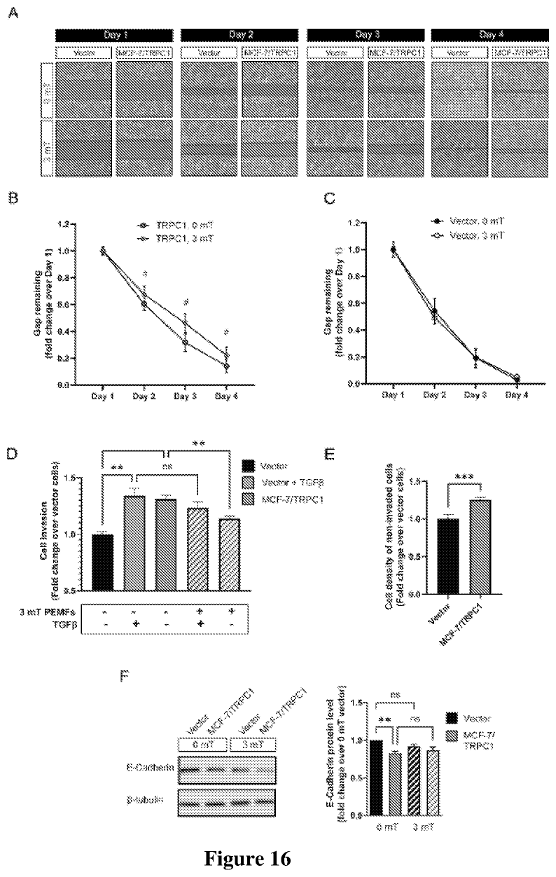

FIG. 17. TRPC1 overexpression sensitizes MCF-7 cells to doxorubicin and PEMF exposure. (A) PEMF and DOX treatment regime for MCF-7/TRPC1 cell DNA quantification. (B) DNA fold change relative to 0 mT vector cells. Statistical analysis was done using the two-sample t-test. (C) Colony formation after 10 days in the presence of 0 nM, 10 nM or 20 nM DOX, without PEMF exposure. Images of colony formation and the quantification of colony size frequency distribution normalized to the total number of colonies in the presence of 10 nM DOX for (D) vector and (E) MCF-7/TRPC1 cells or 20 nM DOX for (F) vector and (G) MCF-7/TRPC1 cells, concomitant with daily PEMF exposure. *, p<0.05, **, p<0.01 and ***, p<0.001 indicate the statistical difference between the respective 0 mT and 3 mT condition within the same bin. (H) PEMF-modulated proliferation of TRPC1-silenced MCF-7 cells 48 h post dsiRNA transfection. Cells were transfected with three independent dsiRNA (green), including a scramble RNA (black). Control (untransfected) MCF-7 cells were exposed to 0 mT (red) or 3 mT (blue) PEMFs. (I) Combined effects of PEMF and DOX treatments on the proliferation of TRPC1-silenced cells. Cells were exposed to PEMFs 24 h post dsiRNA transfection and one day later before DNA content analysis (hatched bars). The data for TRPC1 dsiRNA (green and dark green) was pooled data from two independent TRPC1 dsiRNAs. The statistical analysis was generated using Multiple unpaired t-test for the comparison of two sample means within the same colony size. All experiments were from 3 to 5 independent experiments with *, p<0.05, **, p<0.01, ***, p<0.001, and #, p<0.0001. The error bars represent the standard error of the mean.

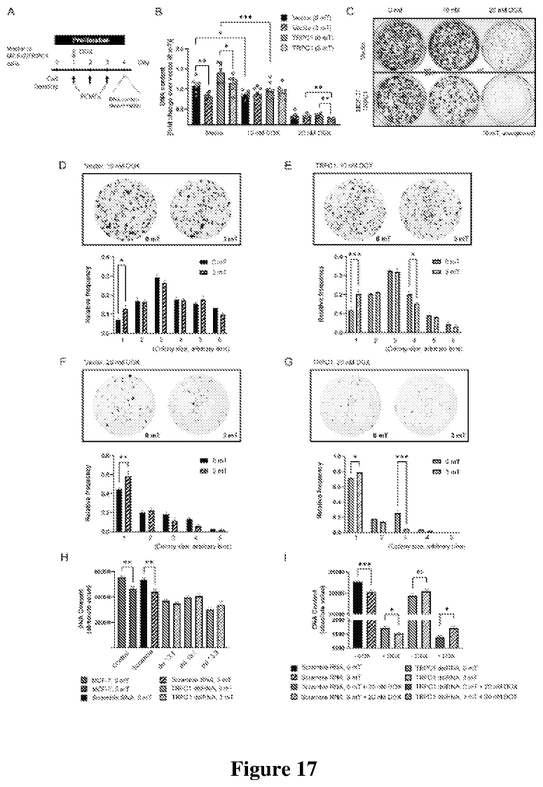

FIG. 18. Schematic representation of cellular events modulated by the overexpression and silencing of TRPC1 gene in MCF-7 breast cancer cells.

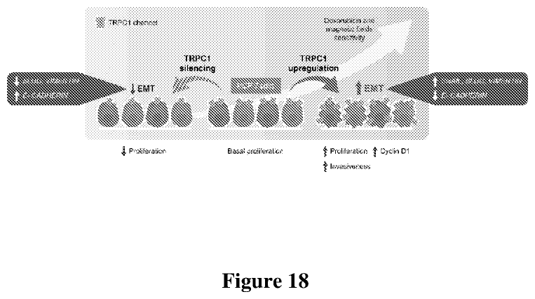

FIG. 19. (A) Illustration of human breast coil prototype. Efficacy validation method of the human breast coil prototype used in tissue culture (B) and mice (C) experiments. (D) Photograph of fully assembled breast coil system to be ultimately employed in human clinical trials. The lumen of the PEMF breast coil was roughly based on the dimensions of existing clinical MRI breast scanning coil devices that are designed to accommodate the human female breast.

FIG. 20. (A) Chemo-sensitivity of MCF-7 cancer cells to Pemetrexed and Cisplatin in combination with PEMF in comparison to DOX. Cells were seeded in 96-well and exposed to 3 mT PEMF for 1 h per day for 3 days. Chemotherapeutic drugs were added (red arrow) on the final day of PEMF before the analysis of DNA content 24 h later. The corresponding bar charts show the absolute DNA content of cells in response to increasing doses of chemotherapeutic agents with or without PEMFs. (B) Representation of a dose-response curve of the same data in (A), treated with DOX, Pemetrexed, and Cisplatin. The X-axis represents log (concentration) and the y-axis represents the absolute intensity of DNA content. Statistical analysis was performed using multiple unpaired t-tests, comparing between 0 mT and 3 mT within each concentration. All experiments were from 3 to 5 independent experiments with *, p<0.05, and #, p<0.0001. The error bars are expressed as the standard error of the mean.

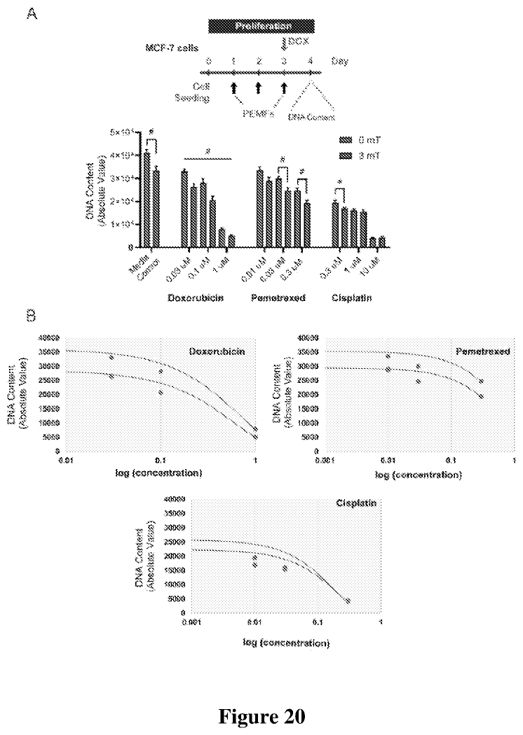

FIG. 21. PEMFs inhibit MDA-MB-231 tumor growth without affecting liver in vivo. (A) Schematic of PEMF exposure of NSG mice implanted with MDA-MB-231 tumors. Implanted tumors were allowed to grow for 3 weeks before the initiation of PEMF exposure once (3 mT×1; 1 h, once a week) or twice (3 mT×2; 1 h once a week for 2 weeks). Flow cytometric analysis was performed 1 week after the last PEMF exposure. Representative scatter dot-plots of B) MDA-MB-231 xenografts and C) mouse livers showing cell population of dissociated tumors sorted based on annexin and propidium iodide staining. The percentages represent the total early and late apoptotic cells.

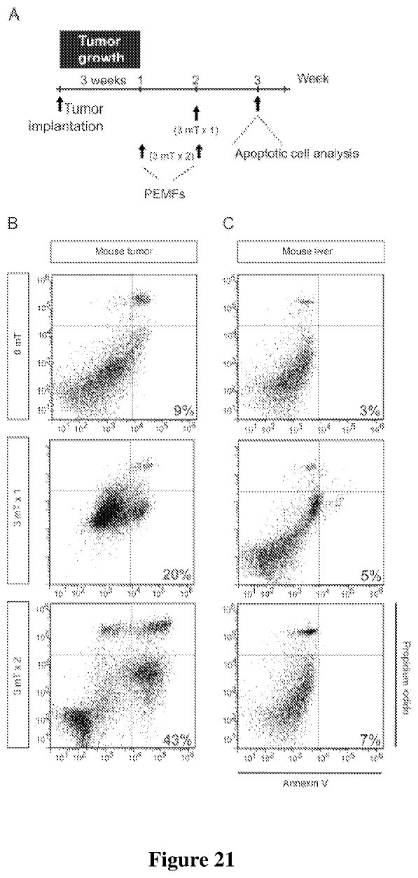

FIG. 22. PEMFs synergize with doxorubicin to inhibit MCF-7 tumor growth in vivo. (A) Schematic of weekly PEMF and DOX exposures on MCF-7 xenograft in mice. Implanted cells were allowed to grow for 3 weeks before the initiation of DOX and/or PEMF treatment. Apoptotic cell determination was performed at the end of the study. (B) Representative scatter dot-plots showing cell population of dissociated tumors sorted based on annexin and propidium iodide staining. Bar charts represent pooled data of early and late apoptotic cell percentages analyzed using flow cytometry. N=6 mice, with *, p<0.05, **, p 1137<0.01, and #, p<0.0001. The error bars are expressed as the standard error of the mean.

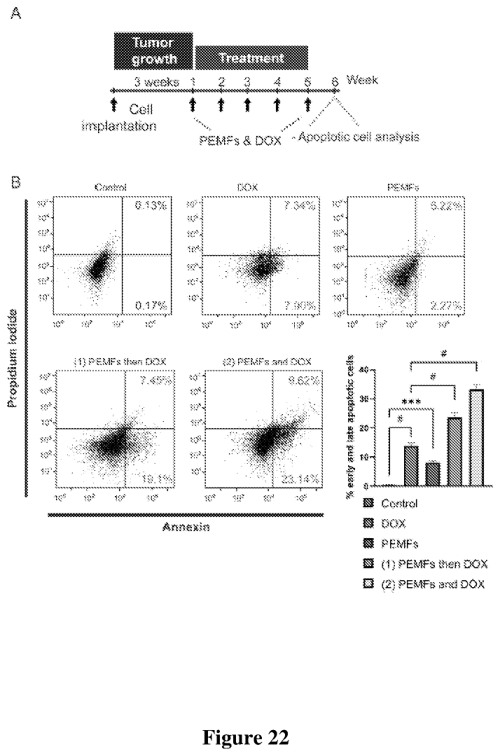

FIG. 23. Recovery of PEMF cytotoxicity upon removal of selective pressure from DOX and reinstatement of TRPC1 expression. Cell proliferation assay using Cyquant DNA content analysis on MCF-7/ADR cell lines, in combination with DOX (100 nM) and PEMF exposure. MCF-7/ADR (96 nM) cells were maintained in 96 nM DOX while MCF-7/ADR (0 nM) were serially passaged in the absence of DOX. Cells were exposed to 3 mT PEMFs for 1 h per day for 3 days. 100 nM DOX was administered 1 h before the final PEMF exposure. Cyquant analysis was performed 24 h after the final PEMF exposure. Statistical analysis was performed using one-way ANOVA with Sidak's multiple comparison test with *, p<0.05, and #, p<0.0001. Data presented were from 2 independent experiments with 6 technical replicates per experiment. The error bars are expressed as the standard error of the mean.

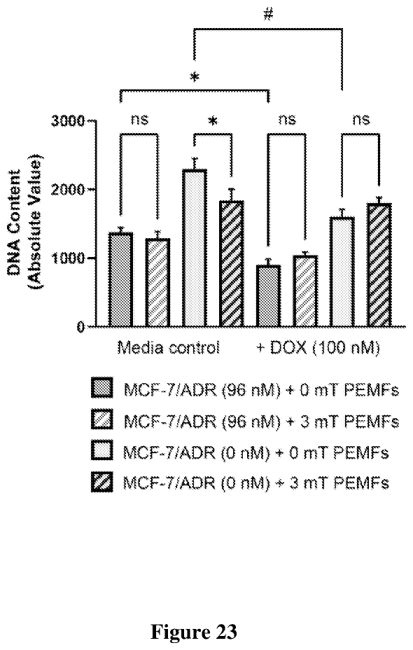

### DETAILED DESCRIPTION

The present invention is directed to cell-derived vesicles as Transient Receptor Potential Channel 1 (TRPC1) channel delivery systems.

The specification teaches a method of preparing cell-derived vesicles comprising TRPC1 protein, the method comprising the steps of a) incubating a population of cells with cytochalasin B and b) isolating the cell-derived vesicles from the population of cells.

Disclosed herein is a method of preparing cell-derived vesicles from a eukaryotic cell, the method comprising:
a) exposing a cell to a pulsed electromagnetic field (PEMF) to induce Transient Receptor Potential Channel 1 (TRPC1) protein expression or activity in the cell;
b) contacting the cell with an actin cytoskeletal disruptor under mixing conditions to promote release of cell-derived vesicles from the cell surface; and
c) isolating the cell-derived vesicles.

The cell-derived vesicles may be used for the recovery of cellular respiratory and proliferative capacities as well as to instil magnetic mitohormetic survival adaptation of a cell.

In one embodiment, the cell-derived vesicles are enriched for TRPC1 protein or a TRPC1-associated protein. The term “TRPC1-associated protein” may refer to any protein that assists in improving TRPC1 protein expression or activity.

Without being bound by theory, the inventors have shown that the simple replacement or supplementation of TRPC1 to quiescent cells (exhibiting low TRPC1 levels) reinstates mitochondrial responses. The inventors were able to achieve this by making membrane vesicles from healthy muscle cells and administering them to deregulated cells.

Cell-derived vesicles are membrane surrounded structures that are released by cells in vitro. Cell-derived vesicles can contain exosomes, proteins, lipids, and nucleic acids and can mediate intercellular communication between different cells, including different cell types, in the body.

Cell-derived vesicles can be isolated from eukaryotic cells. In one embodiment, the cell-derived vesicles are isolated from mammalian cells (such as primate or human cells). In one embodiment, the cell-derived vesicles are isolated from stem cells. Non-limiting examples of such stem cells include adult stem cells, embryonic stem cells, embryonic-like stem cells, neural stem cells, or induced pluripotent stem cells. In some embodiments, the stem cell is an adult stem cell such as a myoblast.

The cells of the present disclosure may optionally be modified, for example, by genetic modification. In some embodiments, the cells are modified to express at least one exogenous nucleic acid and/or at least one exogenous protein. In some embodiments, the cells are modified to express at least one endogenous nucleic acid and/or at least one endogenous protein. The modification may be a transient modification. In other embodiments, the modification may be a stable modification. It is contemplated that by modifying the cells prior to collection of the cell-derived vesicles released by the modified cells, one can collect exosomes containing different amounts and types of proteins, lipids, and nucleic acids as compared to unmodified cells. Any method for cellular modification known to one of skill in the art can be used to modify the cells.

In some embodiments, the cells of the present disclosure are optionally modified to express at least one exogenous or endogenous nucleic acid and/or at least one exogenous or endogenous protein. Non-limiting examples of nucleic acids include one or more or all of DNA and RNA, for example, a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers.

In one embodiment, the method comprises exposing a cell to a pulsed electromagnetic field (PEMF) to induce Transient Receptor Potential Channel 1 (TRPC1) protein expression or activity in the cell.

The expression and efficacy of TRPC1 can be potentiated by prior muscle-specific magnetic field therapy. PEMF exposure may upregulate TRPC1 expression and function when applied to a cell, such as a muscle cell, in vitro. Cell-derived vesicles can therefore be further enriched in TRPC1 expression by only 10 minutes of PEMF exposure at 1.5 mT 12-24 hours before preparation.

In one embodiment, the PEMF is applied at an amplitude of about 1 mT to about 1.5 mT. The PEMF may be applied at about 1 mT, about 1.1 mT, about 1.2 mT, about 1.3 mT, about 1.4 mT or about 1.5 mT. This may provide an optimal induction of TRPC1 protein or activity in the cell.

In one embodiment, the PEMF is a directional PEMF.

The PEMFs may be applied in substantially the same direction as gravity, with further enhancement when the downwardly directed fields are additionally applied orthogonally to a longitudinal axis of the eukaryotic cell.

In one embodiment, the pulsed electromagnetic field (PEMF) is applied orthogonally to a longitudinal axis of the eukaryotic cell in substantially the same direction as gravity (i.e. a downward directional PEMF).

The purified populations of cell-derived vesicles of the present disclosure can be isolated using any method known by those in the art. Non-limiting examples include differential centrifugation by ultracentrifugation, sucrose gradient purification and combination filtration/concentration. In some embodiments, cell debris and other contaminates are removed from the cell-derived vesicle containing fraction

After isolation, the cell-derived vesicles can be concentrated to provide a purified population of cell-derived vesicles. Any appropriate method can be used to concentrate the cell-derived vesicles. Non-limiting examples of such include centrifugation, ultrafiltration, filtration, differential centrifugation and column filtration with a 100 kDA to 300 kDa pore size, or either a 100 kDA to 300 kDa pore size. Further sub-populations can be isolated using antibodies or other agents that are specific for a specific marker expressed by the desired exosome population.

The cell derived vesicles can have a heterogeneous size distribution (from <100 nm up to 1 μm). The method may involve a step of selecting cell derived vesicles of particular sizes, e.g. <100 nm, about 100 nm, about 200 nm, about 300 nm, about 400 nm, about 500 nm, about 600 nm, about 700 nm, about 800 nm, about 900 nm or about 1 μm.

In some embodiments, the methods disclosed herein further comprise formulating the population of cell-derived vesicles by mixing the population with a carrier and/or a therapeutic agent. In some embodiments, the population of cell-derived vesicles comprise proteins, lipids, metabolites, and/or nucleic acids.

In one embodiment, the cell-derived vesicles of the present invention are loaded with myokines, exosomes/microvesicles and/or genetically-engineered agents. The loading of the cell-derived vesicles may be accentuated by PEMF exposure of the correct directionality. PEMF exposure may activate the secretome of cells, such as myoblasts.

PEMF exposure prior to cell-derived vesicles production may, for example, increase their myokine content.

The PEMF treatment may enrich the secretome for anti-cancer myokines, metabolites or factors. In one embodiment, the cell-derived vesicles are further enriched for one or more anti-cancer proteins. These may include HTRA1 and/or KLOTHO.

Exosomes are small lipid-bound, cellularly secreted vesicles that mediate intercellular communication via cell-to-cell transport of proteins and RNA. Exosomes range in size from approximately 30 nm to about 200 nm. Exosomes are released from a cell by fusion of multivesicular endosomes (MVE) with the plasma membrane. Microvesicles, on the other hand, are released from a cell upon direct budding from the plasma membrane (PM). Microvesicles are typically larger than exosomes and range from approximately 100 nm to 1 μm.

Intracellular contents are captured within CDVs upon formation. Muscle cells can be genetically-engineered or chemically-loaded to contain a variety of bioactive agents that will then be captured within CDVs upon their formation for ultimate delivery to recipient cells.

The terms “protein” and “polypeptide” are used interchangeably and refer to any polymer of amino acids (dipeptide or greater) linked through peptide bonds or modified peptide bonds. Polypeptides of less than about 10-20 amino acid residues are commonly referred to as “peptides.” The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

As used herein, the term “modified,” relative to cell-derived vesicles, refers to cell-derived vesicles that have been altered such that they differ from a naturally occurring cell-derived vesicles. Non-limiting examples of a modified cell-derived vesicle include an exosome and/or microvesicle that contains a nucleic acid or protein of a type or in an amount different than that found in a naturally occurring exosome and/or microvesicle.

The term “purified population,” relative to cell-derived vesicles, as used herein refers to plurality of cell-derived vesicles that have undergone one or more processes of selection for the enrichment or isolation of the desired exosome population relative to some or all of some other component with which cell-derived vesicles are normally found in culture media. Alternatively, “purified” can refer to the removal or reduction of residual undesired components found in the conditioned media (e.g., cell debris, soluble proteins, etc.). A “highly purified population” as used herein, refers to a population of cell-derived vesicles in which at least 65%, at least 70%, at least 75%, at least 80%, at least 85%, at least 90%, at least 95%, at least 98%, at least 99% or 100% of cell debris and soluble proteins (e.g., proteins derived from fetal bovine serum and the like) in the conditioned media along with the cell-derived vesicles are removed.

The term “stem cell” refers to a cell that is in an undifferentiated or partially differentiated state and has the capacity to self-renew and to generate differentiated progeny. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, myoblasts, mesenchymal stem cells (MSCs) and neural stem cells (NSCs). In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

In one embodiment, the cells are stem cells. In one embodiment, the cells are myoblasts, dental pulp stem cells or mesenchymal stem cells. In one embodiment, the cells are myoblasts. In one embodiment, the myoblasts are proliferating myoblasts or myoblasts that are at an early expansion stage.

Myoblasts express elevated levels of TRPC1 during early expansion. The method as defined herein may comprise obtaining cell-derived vesicles from proliferating myoblasts that are particularly enriched in TRPC1.

In one embodiment, the cell-derived vesicles are enriched with TRPC1 protein.

In one embodiment, the cells are obtained from a biopsy of a subject. For example, the cells may be obtained via autologous derivatization from a muscle biopsy. Cells can be obtained a few days prior to surgical intervention for CDV production.

In one embodiment, the method comprises contacting the cell with an actin cytoskeletal disruptor under mixing conditions to promote release of cell-derived vesicles from the cell surface. The actin cytoskeletal disruptor may be any actin cytoskeletal disruptor that can be used to disrupt the cytoskeleton structure within a cell.

The actin cytoskeletal disruptor can be a small molecule, a peptide, an antibody, a protein, a nucleic acid (such as an siRNA or shRNA) or a CRISPR-Cas nuclease system. Exemplary agents that disrupt actin polymerization include without limitation cytochalasin B, cytochalasin D, latrunculin A, latrunculin B, migrastatin, gelsolin, chivosazole A, chivosazole F, Clostridium perfringens iota, Clostridium botulinum C2, and desmethoxymajusculamide C.

The phrase “mixing conditions” may refer to, for example, shaking or stirring conditions.

The method as defined herein may comprise obtaining cell-derived vesicles using a modified cytochalasin protocol. The cytochalasin method preferentially vesiculates lipid raft domains, in which TRPC1 is known to perform its physiological role. The lipid raft domains are also very stable, a trait that is conferred to the cell-derived vesicles obtained from lipid rafts domains.

The method may comprise incubating a population of cells with cytochalasin B. The population of cells may be one that has been obtained from a subject. The method may comprise shaking the population of cells with cytochalasin B. The method may comprise shaking the population of cells at about 300 rpm. The shaking may be performed for about 15 mins. The shaking may be performed at about 37° C.

As used herein, the term “subject” includes any human or non-human animal. In one embodiment, the subject is a human. The term “non-human animal” includes all vertebrates, e.g., mammals and non-mammals, such as non-human primates, sheep, dog, cow, chickens, amphibians, reptiles, etc.

Also disclosed herein is a method of preparing cell-derived vesicles from a eukaryotic cell, the method comprising:
a) exposing a cell to cold treatment to induce Transient receptor potential cation channel, subfamily A, member 1 (TRPA1) protein expression or activity in the cell;
b) contacting the cell with an actin cytoskeletal disruptor under mixing conditions to promote release of cell-derived vesicles from the cell surface; and
c) isolating the cell-derived vesicles.

The cold treatment may be treatment with menthol.

Provided herein is a population of cell-derived vesicles obtained according to a method as defined herein.

In one embodiment, the cell-derived vesicles are capable of promoting regeneration of most tissues other than muscle tissues. The cell-derived vesicles may exhibit inherent homing to muscle tissues.

Myoblasts hold the unique capability from other cells types of being fusogenic, a functionality that is conferred by enrichment of phosphatidylserine in the outer leaflet of the surface membrane.

In one embodiment, the cell-derived vesicles as defined herein are fusogenic.

In one embodiment, the cell-derived vesicles are capable of mitochondrial activation

The cell-derived vesicles as mentioned herein may be easily prepared in a short amount of time (e.g. in about 1.5 hours).

The production of the cell-derived vesicles may be highly scalable in that high yields can be produced from muscle progenitor cells during log phase expansion a few days after planting. It is amenable to bench top production. Cell-derived vesicle production may capture overall greater membrane yields, particularly of the surface membrane, which is an important feature in the context of skeletal muscle, as this is where fusogenic capacity lies (high levels of PS in outer leaflet) as well as location of functional TRPC1.

In one embodiment, the cell-derived vesicles have high stability upon production.

In one embodiment, the cell-derived vesicles may escape destruction by immune phagocytes following administration into a subject. This is because the muscle has evolved to escape the “eat me” response, despite having elevated levels of extramembranar phosphatidylserine.

In one embodiment, the cell-derived vesicles may be applied to cancer cells to enhance chemo-sensitivity to an anti-cancer drug. Alternatively, the cell-derived vesicles may be applied to normal cells or tissues to reverse cellular or tissue aging.

The term “oxidative stress” represents an imbalance between the production and manifestation of reactive oxygen species and a biological system's ability to readily detoxify the reactive intermediates or to repair the resulting damage. In humans, oxidative stress contributes to diseases ranging from Alzheimer's, heart disease and stroke to macular degeneration (the leading cause of adult blindness), dry eye, glaucoma and cancer. However, increased oxidative stress also causes an adaptive reaction which produces increased stress resistance and a long-term reduction of oxidative stress in a process known as mitohormesis.

In one embodiment, the cell-derived vesicles may be used to induce magnetic mitohormesis in a subject in the presence of an exogenous magnetic field.

The present disclosure provides purified populations of cell-derived vesicles. In some embodiments, the population of cell-derived vesicles is substantially homogeneous. In other embodiments, the population of cell-derived vesicles is heterogeneous.

The TRPC1 levels and/or magneto-sensitivity of the cell-derived vesicles may be assesses using calcium imaging in suspension. A population of cell-derived vesicles with similar TRPC1 levels and/or magneto-sensitivity may be isolated to obtain a substantially homogenous population of cell-derived vesicles. Alternatively, a heterogeneous population of cell-derived vesicles may be used.

Provided herein is the use of a population of cell-derived vesicles as defined herein for delivering TRPC1 or TRPA1 to a subject.

Provided herein is the use of a population of cell-derived vesicles as defined herein as a biosensor.

In one embodiment, the population of cell-derived vesicles are enriched for TRPC1 and may be used as a biosensor of magnetic field. The level or change in intra-vesicular concentration of calcium in the population of cell-derived vesicles as compared to a reference may be used to detect the presence or strength of a magnetic field.

In one embodiment, the population of cell-derived vesicles are enriched for TRPA1 and may be used as a biosensor of temperature. The level or change in intra-vesicular concentration of calcium in the population of cell-derived vesicles as compared to a reference may be used to provide an indication of the surrounding temperature.

The level or change in intra-vesicular concentration of calcium may be detected with calcium sensing dyes that are well known in the art. The calcium sensing dyes may be loaded into cells prior to production of the cell-derived vesicles.

Provided herein is a pharmaceutical composition comprising cell-derived vesicles obtained according to a method as defined herein.

The pharmaceutical composition may comprise a pharmaceutically acceptable carrier.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, indicator (calcium, pH, voltage, etc.) dyes, wetting or emulsifying agents, pH buffering agents, preservatives, and the like.

Representative pharmaceutically acceptable carriers include any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

Pharmaceutical compositions of the present disclosure may be in a form suitable for administration by injection, in a formulation suitable for oral ingestion (such as, for example, capsules, tablets, caplets, elixirs), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, subcutaneous, intramuscular or intravenous injection.

Provided herein is a method of promoting cellular or tissue regeneration in a subject, wherein the method comprises administering a population of cell-derived vesicles or a pharmaceutical composition as defined herein to the subject.

Provided herein is a method of inhibiting proliferation of a cancer cell in a subject, wherein the method comprises administering a population of cell-derived vesicles or a pharmaceutical composition as defined herein to the subject. The cancer cell may be a TRPC1 expressing cancer cell.

Provided herein is a method of treating a disease or condition in a subject, wherein the method comprises administering a population of cell-derived vesicles or a pharmaceutical composition as defined herein to the subject.

The method may further comprise providing a PEMF therapy to the subject.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either amelioration of, or complete reduction of, one or more symptoms of the disease or condition, or the delaying of the progression of the disease or condition. “Treatment” does not necessarily indicate complete eradication or cure of the disease or condition, or associated symptoms thereof.

In one embodiment, the disease or condition is one that is associated with disruptive metabolism and inflammation. The disease or condition may be cell senescence and disorders associated with metabolic depression. The disease or condition may be disorders associated with enhanced inflammatory and/or metabolic status, such as cancer.

In one embodiment, the disease or condition is cancer or cellular/tissue aging.

The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer.

Provided herein is a method of sensitizing a subject to an anti-cancer therapy, the method comprising administering a population of cell-derived vesicles or a pharmaceutical composition as defined herein to the subject in combination with the anti-cancer therapy.

In one embodiment, the subject is suffering from a cancer. The cancer may be any cancer that is characterised by elevated TRP channel expression, in particular TRPC1. The cancer may be breast cancer, prostate cancer or liver cancer. The cancer may be a metastatic cancer.

In one embodiment, the anti-cancer therapy is a radiotherapy chemotherapy or PEMF treatment.

In one embodiment, the anti-cancer therapy is PEMF therapy.

In one embodiment, the anti-cancer therapy is a chemotherapy. The method may further comprise PEMF therapy.

In one embodiment, the anti-cancer therapy is a combination of PEMF therapy and chemotherapy.

In one embodiment, the anti-cancer therapy is a combination of PEMF therapy and doxorubicin treatment.

The term chemotherapy may refer to treatment with a chemotherapeutic agent. Examples of chemotherapeutic agents include antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, calmodulin inhibitors, chemotherapy sensitizers, duocarmycins, etoposides, fluorinated pyrimidines, ionophores, lexitropsins, maytansinoids, nitrosoureas, platinols, pore-forming compounds, purine antimetabolites, puromycins, radiation sensitizers, rapamycins, steroids, taxanes, topoisomerase inhibitors, vinca alkaloids, or the like.

In one embodiment, the chemotherapeutic agent is an anthracycline. The anthracycline may be daunorubicin, doxorubicin, epirubicin, idarubicin, mitoxantrone or valrubicin. In one embodiment, the chemotherapeutic agent is doxorubicin.

The chemotherapeutic agent may be administered at a dose of about 5 mg/kg, about 10 mg/kg, about 15 mg/kg, about 20 mg/kg, about 25 mg/kg, about 30 mg/kg, about 35 mg/kg or about 40 mg/kg.

The PEMF therapy may be an exposure of about 1 mt, about 2 mT, about 3 mT, about 4 mT or about 5 mT of PEMF. This may be administered for about 30 min, about 1 h about 1.5 h, about 2 h, about 2.5 h or about 3h. The PEMF therapy may be administered 1, 2, 3, 4, 5, 6 or 7 times per week. In one embodiment, the PEMF therapy is administered at about 3 mT for 1 h, once a week.

The PEMF therapy and the chemotherapy may be either administered concurrently or sequentially. In one embodiment, the PEMF therapy is administered prior to or following the chemotherapy.

The population of cell-derived vesicles or pharmaceutical composition may be administered prior to or following an anti-cancer therapy. The population of cell-derived vesicles or pharmaceutical composition may also be administered concurrently with the anti-cancer therapy.

Provided herein is a method of inhibiting proliferation of a cancer cell in a subject, wherein the method comprises administering a PEMF therapy to the subject. The cancer cell may be a TRPC1 expressing cancer cell. The method may further comprising administering a chemotherapeutic, such as doxorubicin, to the subject.

Provided herein is a method of treating a cancer in a subject, wherein the method comprises administering a PEMF therapy to the subject. The method may further comprising administering a chemotherapeutic, such as doxorubicin, to the subject. The cancer may be a TRPC1 expressing cancer.

Provided herein is a method of sensitizing a subject to an anti-cancer therapy, the method comprising administering a PEMF therapy to the subject in combination with the anti-cancer therapy. The anti-cancer therapy may be a chemotherapeutic, such as doxorubicin. The subject may be suffering from a TRPC1 expressing cancer.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1

Cell Culture:

Otherwise stated, all reagents for cell culture experiments were purchased from Thermo Fischer Scientific, Switzerland. Murine skeletal muscle myoblasts (C2C12; ATCC, USA) were cultured in DMEM containing glucose (4.5 g L⁻¹) and sodium pyruvate (1 g L⁻¹) and supplemented with 5% fetal bovine serum and L-glutamine (2 mmol L⁻¹) in a humidified atmosphere at 37° C. and 7% CO₂. Cell confluence was kept between 20% and 40% to prevent premature differentiation and maintain TRPC1 expression at highest levels. For myotube induction, myoblasts were grown to >80% confluence for 3 days prior to the provision of DMEM supplemented with only 2% horse serum for 2 days.

Cell-Derived Vesicle (CDV) Production

In preparation for CDV production, 75 cm² culture flasks of myoblasts or myotubes, after 24 h of seeding or 48 h of differentiation induction, respectively, were washed once with phosphate buffered saline (PBS) pH 7.4, incubated for 30 s with trypsin at room temperature (TrypLE express), and then incubated in serum-free RPMI 1640 medium supplemented with cytochalasin B (10 μmol L⁻¹; Sigma, Switzerland) for 15 min. The culture flask was mounted onto an incubation shaker (KS 4000 i control, IKA, Germany) set to 300 rpm at 37° C. to increase vesicle formation yield during the incubation with cytochalasin B. The supernatant was subsequently collected and centrifuged at 700 g for 5 min to separate cells and cell debris from the CDV solution. The supernatant was stored at 4° C. until use.

Pulsing Magnetic Field (PEMF) Exposure System:

A barrage of magnetic pulses of 6 ms duration is applied to cells or animals at a repetition rate of 15 Hz and at flux densities between 1-4 mT. Each 6 ms burst consisted of a series of 20 consecutive asymmetric pulses of 150 μs on and off duration with an approximate rise time of 17 T/s. The background magnetic flux density measured in the chamber is below 1 μT between 0 Hz to 5 kHz. The coil size, position and individual number of windings were numerically optimized by a CST low frequency solver for low field non-uniformity over a wide frequency range taking into consideration the shielding capacity of the μ-metallic chassis. The measured field non-uniformity did not exceed 4% within the uniform exposure region of the coils.

Engineering expertise for the designing and fabrication of magnetic exposure systems can be taken over by QuantumTx

### Example 2

Assessment of CDV content and function after PEMF exposure

Cell Culture

C2C12 myoblasts were seeded at a density of 600,000 cells per T75 cm² flask. The cells were allowed to grow for another 24 h in DMEM media supplemented with 10% FBS without antibiotics. Two hours prior to magnetic stimulation, the media was changed to DMEM without FBS and antibiotics. Six hours after magnetic stimulation at 1.5 mT for 10 min, the exposed and non-exposed (0 mT control flask) were subjected to CDV isolation protocol.

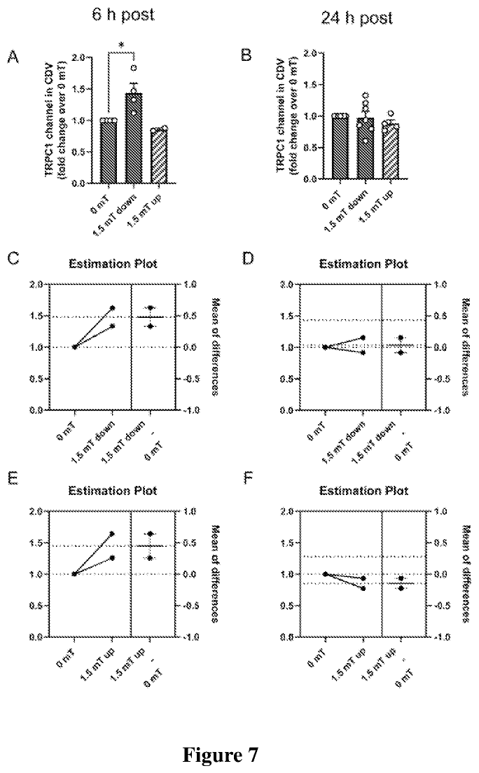

For the analysis of C2C12 myoblast proliferation, recipient cells were seeded at a density of 3000 cells per cm² in a 6-well plate. 24 h later, CDVs from donor myoblasts were reconstituted in 2 ml fresh DMEM containing 5% FBS and given to the plated recipient cells. The recipient cells were allowed to grow for another 24 h and cell number enumerated using standard Tryphan Blue exclusion assay (FIG. 7C to 7F).

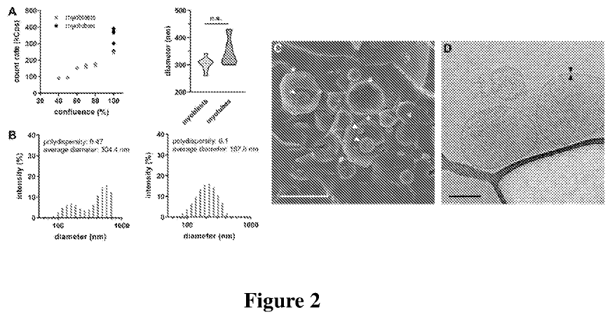

For the analysis MCF-7 breast cancer cells proliferation in the presence of recombinant HTRA1 (Cusabio, mouse recombinant HTRA1, partial) (FIG. 2E) or secreted HTRA1 donor conditioned media (FIG. 2F), MCF-7 cancer cells were seeded at 2000 cells per well of a 96-well plate using RPMI media containing 10% FBS without antibiotics. 24 h later, recombinant HTRA1 or conditioned media were given to the cells and incubated for 48 h before the assessment of proliferation using Cyquant DNA content assay kit.

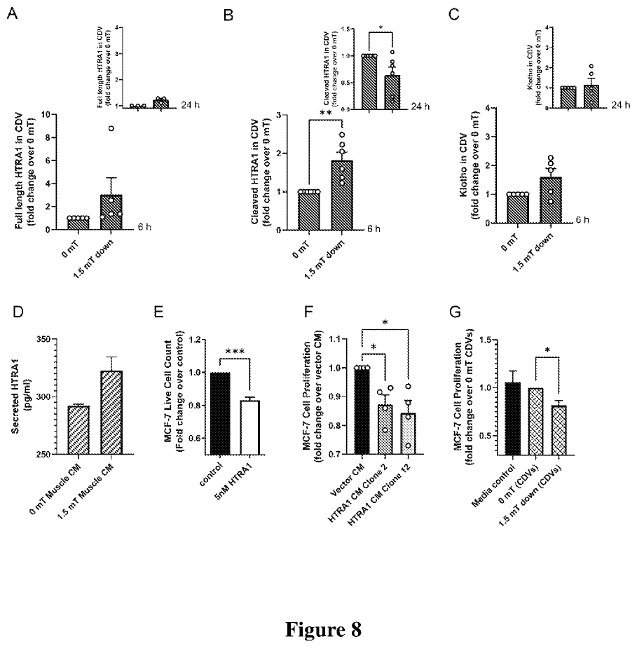

The secreted HTRA1-conditioned media were collected from stable MBA-MD-231 breast cancer cells overexpressing human HTRA1 in a p2A-GFP plasmid (FIG. 8F). The stable cells were generated using Geneticin (GIBCO) antibiotic pressure as well as single cell GFP positive sorting using flow-assisted sorter (MoFlo, Cell Sorter, Beckman Coulter). Clones 2 and 12 were HTRA1 overexpressing clones with stable HTRA1 expression in the cell as well as in the conditioned media.

Western Analysis

Isolated CDVs were lysed in 70 ul of RIPA buffer containing protease inhibitor, respectively. Protein concentration was determined using standard BCA assay. 20 ug of protein for each sample was resolved in a denaturing and reducing SDS-PAGE gel and transferred onto PVDF membrane for subsequent antibody probing. The abundance of TRPC1, HTRA1 and Klotho proteins in the CDVs was estimated using LICOR Odyssey FC Imaging System. Primary antibodies and their dilution used in the study were as follow: TRPC1 (Santa Cruz; 1:300), HTRA1 (Proteintech, 1:1000) and Klotho (Abcam; 1:1000). Secondary antibodies used were anti-mouse (1:10,000) and anti-rabbit (1:3000) antibodies from Bio Rad Laboratories.

ELISA

For the analysis of secreted HTRA1 in the media of C2C12 myotubes, myoblasts were differentiated into myotubes according to Yap et. al (2019) with some modifications. Briefly, myoblasts were seeded at a density of 6000 cells per cm² in a T75 flask. 3 days later, media were replaced with DMEM containing 2% horse serum without antibiotics to promote the differentiation of myoblasts. The media were replenished again 2 days later. On Day 7, the media of the differentiated myotubes were replaced with DMEM without FBS and antibiotics and subjected to 0 mT or 1.5 mT magnetic stimulation. The conditioned media were collected 24 h later for ELISA analysis for the abundance of HTRA1 protein (Novus Biologicals). The ELISA plate was read using Cytation 5 Reader.

### Example 3

Materials and Methods

MCF-7 Breast Cancer Xenograft and Patient-Derived Xenograft (PDX) Model in NSG Mice

NSG (NOD·Cg-Prkdc^{scid} Il2rg^{tm1Wjl}/SzJ) mice, which lack human-specific cytokines and human leukocyte antigen (HLA) expression on stromal cells were used to host the breast cancer cell lines or patient-derived xenografts (PDX) (22). The NSG mice were purchased from Jackson's laboratory and used at 8-10 weeks of age. Briefly, each female NSG mouse was implanted with a subcutaneous 60-day (0.36 mg) slow-release estradiol pellet (Innovative Research of America). Each patient tumor was equally divided into 5 chunks and implanted into the dorsal flank of 5 animals corresponding to the 5 different treatment groups. The tumors were allowed to grow for 3 weeks. For MCF-7, 1×10⁶ cells were counted and mixed in a 40-60% ratio with Matrigel Growth Factor (Bio Laboratories, Cat No. 354230). The cells are injected subcutaneously into the dorsal flank of the animals corresponding to the different treatment groups. The animals were given 20 mg/kg DOX intravenously and/or PEMFs stimulation for 1 h weekly for 5 weeks. At the end of the study, tumor volume was measured and isolated for apoptotic cell determination.

Chick Chorioallantoic Membrane (CAM) Model

The chick chorioallantoic membrane (CAM) assay (23) was performed using fertilized Bovans Goldline Brown chicken eggs purchased from Chew's Egg Farm Pte Ltd., Singapore. Briefly, eggs were placed horizontally in a 38.5° C. humified chamber of 70% humidity for 3 days. On day 3, 3 to 4 ml of albumin was removed through a hole in the apex of the eggs using an 18G needle on a 5 ml syringe to lower the CAM. An oval 1 cm² hole was then made on the center of the eggs and covered using a 1624W Tegaderm semi-permeable membrane. On day 7, the eggs were inoculated with 1.5×10⁶ MCF-7 cells resuspended in 50 μl of Matrigel (Sigma Aldrich) on the blood vessel of the CAM. Prior to the inoculation of the MCF-7 cells, the blood vessels closer to the CAM were gently perforated using a dry glass rod. The eggs were resealed using Tegaderm and left for another 3 days. The eggs were then exposed to PEMF stimulation on days 10, 11, and 12 for 1 h each day. Tumor weight was determined on Day 14 and subsequently processed for Western analysis. For the administration of DOX, DOX in saline at a concentration of 0.04 μg per gram of egg was prepared in a total volume of 20 μl, and added onto a small sterile filter paper placed on the CAM vessel next to the tumor. DOX was added 1 h prior to the first PEMF exposure.

Histological Analysis and TUNEL Assay

Isolated chicken embryo liver tissues were fixed in 2.5% PFA and 15% sucrose in PBS for 24 h at 4° C. Tissues were embedded in cryoprotectant Tissue-Tek® O.C.T. Compound and were sectioned at 10 μM thickness. TUNEL assay was performed using Click-iT Plus TUNEL Assay kit (Thermo Fisher Scientific) according to manufacturer's protocol. For human breast biopsies, the tissues were kept in RPMI media supplemented with 10% FBS and exposed to 3 mT PEMF for 1 h. They were maintained in a standard tissue culture incubator for 24 h before fixation using 4% PFA overnight, which were subsequently processed and embedded in paraffin blocks. Tissue biopsies were sectioned at 5 μm thickness and stained with In Situ Cell Death Detection Kit (Roche) as per manufacturer's instruction. The stained sections were viewed using Olympus FV1000 fluorescence microscope.

Cell Culture and Pharmacology

MCF-7 (HTB-22™) cells were acquired from American Type Culture Collection (ATCC) and maintained in RPMI (Gibco) supplemented with 10% FBS (Hyclone) and maintained in a humidified incubator at 37° C. in 5% CO2. MDA-MB-231 cells were a kind gift from Dr. Glenn Bonney, NUS and were authenticated by ATCC using human STR (short tandem repeat) profiling. MCF10A cells were acquired from Dr. Andrew Tan's laboratory (NTU). MDA-MB-231 cells were maintained in DMEM (Gibco) and 10% FBS. MCF10A cells were maintained in growth media containing DMEM/F12 (Gibco) supplemented with 5% horse serum (Hyclone), 20 ng/ml EGF (Peprotech), 0.5 mg/ml Hydrocortisone (Sigma), 100 ng/ml cholera toxin (Sigma) and 10 μg/ml insulin (Sigma). Cells were trypsinized and passaged every 3 days using TrypLE Express reagent (Gibco). MCF-7/ADR cells resistant to 96 nM DOX were generated using a progressive incubation of cells in low 0.3 nM up to 96 nM DOX over 4 months. The concentration of DOX was doubled weekly upon cell reseeding. Doxorubicin hydrochloride (DOX) (Abcam, ab120629) was reconstituted in DMSO to make a stock concentration of 25 mM and stored at −80° C. Subsequent dilutions of DOX were made in distilled water to keep DMSO concentration below 0.01%. No cell culture antibiotics were used throughout the experiments.

Cell Count and DNA Content Analysis

For cell enumeration using trypan blue exclusion assay, MCF-7, MDA-MB-231, or MCF10A cells were seeded at 6000 cells/cm² per well of a 6-well plate. For MCF10A cells, they were plated in growth media without EGF. Cell counting was performed using 3 wells of a 6-well plate for technical replication. For DNA content analysis using Cyquant cell proliferation assay (Invitrogen), cells were seeded at 2000 cells per well and performed with 8 technical replicates in a 96-well plate. Seeded cells were left for 24 h before treatment with DOX or exposed to PEMFs. Cyquant stained DNA was measured using at 480/520 nm using Cytation 5 microplate reader (BIOTEK).

Clonogenic Assay and Quantification of Colonies

In vitro clonogenic assay was performed using crystal violet staining (24). Briefly, MCF-7 cells were seeded either at 100 or 1000 cells per well of a 6-well plate. The cells were treated with DOX on Day 1, 4, and 7 in RPMI supplemented with 10% FBS. 3 mT PEMFs stimulation was administered for 1 h from Day 1 to Day 10. On Day 11, the cells were rinsed in PBS and stained with crystal violet stain consisting of 0.5% crystal violet and 25% glutaraldehyde (Sigma Aldrich) in distilled water for 3 h. Stained colonies were rinsed with 2 changes of tap water and left to dry. Images of the colonies were taken using Chemidoc Imaging System (BIORAD) under the Coomassie Blue Stain filter setting. The number of colonies and colony size per well was estimated using the ImageJ Analyze particle option using 3 to 3500-pixel unit with a circularity of 0.2 to 1. The mean survival factor (colony count) was determined as the number of surviving cells over the number of cells plated and normalized to the survival factor of the control group expressed as fold change. The colony size relative frequency was determined by binning colonies into several bins, according to their relative size from the smallest to the largest colonies after normalizing to the total number of cells.

Reactive Oxygen Species Analysis Using DCH2FDA

Cells were seeded in 96-well clear bottom black well (Costar) at a density of 10,000 cells per well at 8 replicates per condition and left to settle for 24 h before commencing the experiment. Cells were rinsed with warm phenol-free and serum-free (PFSF) RPMI (GIBCO) and incubated with PFSF RPMI containing 10 μM DCH2FDA (Invitrogen) for 30 min in a standard tissue culture incubator. The dye was then rinsed out using warm PFSF RPMI and treated with tert-Butyl hydroperoxide (TBH, 1 mM; Sigma Aldrich) or 50 μM DOX (Abcam) in PFSF RPMI. Cells were immediately exposed to 10 min of 0 mT or 3 mT PEMFs exposure before proceeding to ROS determination using a Cytation 5 microplate reader (BIOTEK) at Ex/EM: 492/520 nm for 20 min with the temperature set at 37° C.

Western Analysis

Cell lysates were prepared in ice-cold radioimmunoprecipitation assay (RIPA) buffer containing 150 mM NaCl, 1% Triton X-100, 0.5% sodium deoxycholate, 0.1% SDS and 50 mM Tris (pH 8.0) supplemented with protease and phosphatase inhibitors (PhosphoSTOP, Roche). Cells were lysed for 20 min and centrifuged for 10 min at 13,500 rpm. The protein concentration of the soluble fractions was determined using a BCA reagent (Thermo Fisher Scientific). 25-50 μg of total protein was resolved using 10% or 12% denaturing polyacrylamide gel electrophoresis and transferred to PVDF membrane (Immobilon-P, PVDF). Proteins on PVDF membranes were blocked using 5% low-fat milk in TB ST containing 0.1% Tween-20 and incubated with the primary antibody in SuperBlock TBS (Thermo Fisher Scientific) overnight at 4° C. The primary antibodies used were: TRPC1 (1:300; Santa Cruz), Cyclin D1 (CD1, 1:300; Santa Cruz), GFP (1:1000; Proteintech), β-actin (1:10,000; Proteintech), a-tubulin (1:5000; Proteintech). The membranes were washed in TB ST. Anti-rabbit or anti-mouse antibody conjugated to horseradish peroxidase (HRP) were diluted (1:3000, Thermo Fisher Scientific) in 5% milk in TBST and were incubated with the membranes for 1 h at room temperature. The membranes were incubated in WestPico or WestFemto chemiluminescent substrate (Thermo Fisher Scientific), detected and analyzed using LI-COR Image Studio.

Laser Confocal Imaging

For the visualization of GFP and Vimentin abundance in TRPC1 overexpressing MCF-7 cells, the cells were seeded onto coverslips at a density of 100,000 cells per well of a 6-well plate (NUNC). 24 h post-seeding, the cells were rinsed with PBS and fixed in 4% paraformaldehyde for 20 min. For the direct visualization of the expression of GFP in vector-only and MCF-7/TRPC1 cells, the cells on coverslips were mounted onto glass slides using ProLong Gold Antifade Mountant (Thermo Fisher Scientific). The cells were then analyzed using the Olympus FV1000 confocal laser scanning microscope. For the visualization of VIMENTIN, the cells were permeabilized with 0.1% Triton in PBS for 10 min after fixation. The cells were then blocked in SuperBlock TBS (Thermo Fisher Scientific) followed by Vimentin antibody (Santa Cruz, 1:100) incubation overnight, followed by secondary Alexa Fluor 594 antibody (1:500, Thermo Fisher Scientific) for 1 h at room temperature. Washes between steps were done with PBS with 0.1% Tween (Sigma Aldrich). Nuclei of cells were co-stained with DAPI (Sigma Aldrich) for 10 min Cells were finally mounted and visualized using a laser scanning confocal microscope. For the quantitative analysis of Vimentin abundance, the total absolute intensity per view was normalized to the number of nuclei to yield a mean protein intensity per cell. The average of the mean protein intensity per cell (at least 10 cells per view) from multiple replicates were used to compute and compare the abundance of Vimentin protein between vector-only and MCF-7/TRPC1 cells.

Real-Time qPCR and TRPC1 Silencing

Quantitative reverse-transcription polymerase chain reaction (RT-qPCR) was carried out using the SYBR green-based detection workflow. Briefly, total RNA was harvested from MCF-7 cells using Trizol reagent (Thermo Fisher Scientific) and 0.5 μg of RNA was reverse transcribed to cDNA using iScript cDNA Synthesis kit (Bio-Rad). Quantification of gene transcript expression was performed using SSoAdvanced Universal SYBR Green (Bio-Rad) on the CFZ Touch Real-Time PCR Detection System (Bio-Rad). Relative transcript expression was determined using the 2-ΔΔCt method, normalized to β-actin transcript levels.

The qPCR primers used were: TRPC1, F: 5′-AAG CTT TTC TTG CTG GCG TG (SEQ ID NO: 1), R: 5′-ATC TGC AGA CTG ACA ACC GT (SEQ ID NO: 2); SNAIL, F: 5′-CGA GTG GTT CTT CTG CGC TA (SEQ ID NO: 3), R: 5′-CTG CTG GAA GGT AAA CTC TGG A (SEQ ID NO: 4); SLUG, F: 5′-TAG AAC TCA CAC GGG GGA GAA G (SEQ ID NO: 5), R: 5′-ATT GCG TCA CTC AGT GTG CT (SEQ ID NO: 6); VIMENTIN F: 5′-AAG GCG AGG AGA GCA GGA TT (SEQ ID NO: 7), R: 5′-AGG TCA TCG TGA TGC TGA GA (SEQ ID NO: 8); and β-actin, 5′-AGA AGA TGA CCC AGA TCA TGT TTG A (SEQ ID NO: 9), R: 5′-AGC ACA GCC TGG ATA GCA AC (SEQ ID NO: 10).

For TRPC1 silencing in MCF-7 cells, two pre-designed dicer-substrate short interfering RNAs (dsiRNA, IDT) were used to knock down the expression of TRPC1. Both dsiRNAs targeted the coding-sequence of TRPC1 (NM_001251845). Transfection of dsiRNA was performed using Lipofectamine 3000 reagent (Invitrogen) as per manufacturer's protocol. TRPC1-silenced cells were validated using qPCR 48 h post dsiRNA transfection using primers against TPRC1, SNAIL, SLUG and VIMENTIN as indicated above, relative to cells transfected with scramble dsiRNA.

Migration Assay

MCF-7 cells at a density of 30,000 cells in 120 μl RPMI supplemented with 10% FBS were seeded into each gap of a 4-well 3.5 mm culture dish insert (ibidi). The cells were left to adhere for 24 h before the removal of the insert and the addition of RPMI media containing 10% FBS to a total volume of 2 ml per dish. Closure of the gaps was captured using light microscopy on all four limbs of the insert, taken every 24 h. The average of 16 gap distances was considered from the 4 limbs with 4 readings arising from each limb. The images of the gap distances were analyzed using ImageJ.

Invasion Assay

Invasion assay was performed using the CytoSelect 24-well Cell Invasion Assay kit (Cell Biolabs, Inc.) according to the manufacturer's protocol. Briefly, 300,000 cells were seeded in the cell culture insert after the rehydration of the basal membrane in FBS-free RPMI media. The lower well of the invasion plate was filled with RPMI media supplemented with 10% to promote the invasion of cells through the basal membrane. 20 ng/ml TGFβ was added to selected conditions in the cell culture insert to stimulate cell invasion. The setup was incubated for 48 h in a standard tissue culture incubator before the extraction and staining of the invaded cells from the basal membrane. The lysates from the extracted cells were analyzed at OD 560 using Cytation 5 microplate reader (BIOTEK).

Generation of Plasmid and Stable Cell Line

GFP-TRPC1 plasmid was generated by PCR amplification of full-length human TRPC1 cDNA (Accession: NM_001251845.2; 2382 base pairs) and directionally subcloned into the pEGFP-C1 vector. Transfection of plasmids in MCF-7 cells was carried out using Lipofectamine 3000 reagent (Invitrogen). 48 h after plasmid transfection, stable cells were selected in RPMI containing 750 μg/ml Geneticin (Invitrogen), 10% FBS, and 1% Pen/Strep (Gibco) in 5% CO2 at 37° C. GFP vector and GFP-TRPC1 cells were enriched for GFP positive cells using Beckman Coulter Moflo Astrios cell sorter. Stables cells were subsequently maintained in complete RPMI media containing 500 μg/ml Geneticin. The overexpression of GFP-TRPC1 in the stable cells was characterized using qPCR, immunofluorescence, and western analysis. GFP stable cells are referred to as vector-only cells while GFP-TRPC1 overexpression stables cells are referred to as MCF-7/TRPC1 cells.

Apoptotic Assay

For apoptotic cell determination, the tumors were dissociated to single cells using the MACS Tumor Dissociation Kit in combination with the gentleMACS Dissociator (Miltenyi Biotec) as according to the manufacturer's protocol. After dissociation, the cells were filtered through a 30 μm MACS SmartStrainer. Cells were pelleted from the filtrate at 300 g×7 min and resuspended in 400 μl Binding Buffer. The cells were incubated with Annexin V FITC and Propidium Iodide (Sigma Aldrich) for 15 min in the dark at room temperature. After incubation, the cells were pelleted and resuspended in 100 μl Binding Buffer for analysis by flow cytometry using BD Accuri C6 cytometer (BD Biosciences, CA, USA).

Breast PEMF Coil

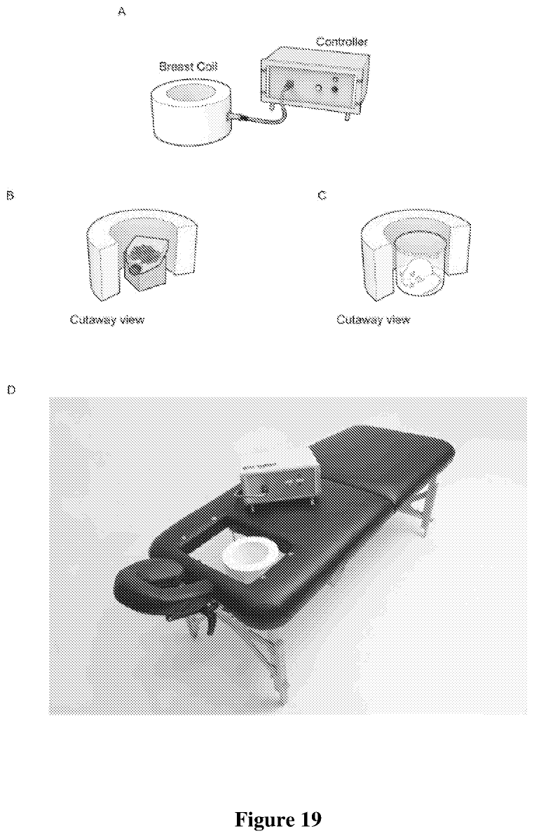

The breast coil design is based on a classical Helmholtz-coil configuration optimized for field uniformity within the dimensions of 120 mm height and 75 mm of radius (FIG. 19A to 19C). The coil dimensions were derived from existing clinical MRI breast scanning coils. The coil is accommodated in a standard patient bed to allow for comfortable positioning of the patients during the entire course of the exposure session (FIG. 19D).

The breast coil system was contract fabricated by Hex Ltd. (Singapore) in accordance with our specifications and consists of a field applicator module (field generating coil) and a power amplifier module. A proprietary coil configuration was unitized for optimal signal generation within the field applicator. A precision wire winding process ensured the generation of a uniform electromagnetic signal within the field applicator volume.

The amplifier module supports the power requirement for the field applicator to generate specified pulsed electromagnetic using a firmware fine-tuned with minimal heat dissipation, ensuring that the various signal output specifications are within defined tolerances. A power and current consumption safety monitoring module is designed to monitor current consumption of the field applicator in real-time with a feedback mechanism to the micro-controller. The system allows active interruption of treatment in the event of current over-flow or excessive field exposure to the subject using the device. The biological efficacy of the system was validated in a subset of the cell and animal experiments (FIG. 19A to 19C).

Statistical Analysis

All statistics were carried out using GraphPad Prism (Version 9) software. One-way analysis of variance (ANOVA) was used to compare the values between two or more groups supported by multiple comparisons. This was followed by Bonferroni's posthoc test. For the comparison between two independent samples, the Student's t-test was performed.

### REFERENCES

Crocetti S et al (2014) Impedance flow cytometry gauges stem cell proliferative capacity by detecting TRPC1 expression. Cytometry A 85A: 525-536. doi: 10.1002/cyto.a.22461
Yap J L Y at al. (2019) Ambient and supplemental magnetic fields promote myogenesis via a TRPC1-mitochondrial axis: evidence of a magnetic mitohormetic mechanism. FASEB Journal 13 (11): 12853-12872. doi.org/10.1096/fj.201900057R
Parate D et al (2020) Pulsed electromagnetic fields potentiate the paracrine function of mesenchymal stem cells for cartilage regeneration. Stem Cell Research & Therapy 11 (1): 46. doi.org/10.1186/s13287-020-1566-5
Tai Y K et al (2020) Magnetic fields modulate metabolism and gut microbiome in correlation with PGC-1α expression: follow-up to an in vitro magnetic mitohormetic study. FASEB Journal 34 (8): 11143-11167. doi:10.1096/fj.201903005RR

1. A method of preparing cell-derived vesicles from a eukaryotic cell, the method comprising:
a) exposing a cell to a pulsed electromagnetic field (PEMF) to induce Transient Receptor Potential Channel 1 (TRPC1) protein expression or activity in the cell;
b) contacting the cell with an actin cytoskeletal disruptor under mixing conditions to promote release of cell-derived vesicles from the cell surface; and
c) isolating the cell-derived vesicles.

2. The method of claim 1, wherein the cell-derived vesicles are enriched for TRPC1 protein and/or TRPC1-associated protein(s).

3. The method of claim 1, wherein the eukaryotic cell is a stem cell.

4. The method of claim 3, wherein the stem cell is a myoblast.

5. The method of claim 1, wherein the PEMF is applied in substantially the same direction as gravity.

6. The method of claim 1, wherein the PEMF is applied orthogonally to a longitudinal axis of the eukaryotic cell in substantially the same direction as gravity.

7. The method of claim 1, wherein the PEMF is applied at an amplitude of about 1 to about 1.5 mT.

8. The method of claim 1, wherein the eukaryotic cell is in a log-phase growth stage.

9. The method of claim 1, wherein the cell is cultured in the absence of aminoglycoside antibiotics.

10. The method of claim 1, wherein the cell-derived vesicles are further enriched for one or more anti-cancer proteins.

11. A population of cell-derived vesicles obtained according to a method of claim 1.

12. The population of cell-derived vesicles of claim 11, wherein the population is used for delivering TRPC1 or TRPC1-associated proteins to a subject.

13. The population of cell-derived vesicles of claim 12, wherein the subject is suffering from a disease or condition that is associated with disruptive metabolism and inflammation.

14. The population of cell-derived vesicles of claim 12, wherein the subject is suffering from a disease or condition that is cancer or cellular/tissue aging.

15. The population of cell-derived vesicles of claim 11, wherein the population is used as a biosensor.

16. A pharmaceutical composition comprising cell-derived vesicles obtained according to a method of claim 1.

17-25. (canceled)
